(19) United States
(12) Patent Application Publication
(10) Pub. No.: US 2025/0099421 A1
(43) Pub. Date: Mar. 27, 2025

(54) Analgesic Therapeutics Based on conjugation of Biocompatible Polymers to Ion Channel Modulators

(71) Applicant: Relevium Medical, Galway (IE)

(72) Inventors: Alison Liddy, Galway (IE); Christophe Labriere, Galway (IE); Mehnaz Ferdousi, Galway (IE); Sahar Avazzadeh, Galway (IE); Simona Cacciapuoti, Galway (IE)

(21) Appl. No.: 18/475,687

(22) Filed: Sep. 27, 2023

(51) Int. Cl. A61K 31/357; A61K 9/00; A61K 31/165; A61K 47/61; A61P 19/02

(52) U.S. Cl. CPC A61K 31/357; A61K 9/0019; A61K 31/165; A61K 47/61; A61P 19/02

(57) ABSTRACT

Compositions and methods for providing sustained and long lasting release of ion channel modulators as analgesics are provided. The compositions comprise a carrier, and a polymer covalently linked to at least one class of ion channel modulators. The ion channel modulators are selected from the group consisting of modulators of transient receptor potential cation channel subfamily V member 1 (TRPV1) channels, voltage gated sodium (Nav) channels, voltage gated calcium (Cav) channels, and combinations thereof. The polymers in the compositions are biocompatible, for example hyaluronic acid or polyethylene glycol. The covalent bond between the ion channel modulator and the polymer may be direct or via a linker. The compositions of this disclosure can be administered as injections locally. The compositions of this invention provide pain relief with a reduced initial acute period of sensitisation when applied to pain sensing neurons.

### FIELD OF INVENTION

This invention relates to compositions and methods related to the modulation of excitable tissue activity. In particular these tissues include nerves, and the compositions and methods are specifically for use in conditions including pain or other cases where there is inappropriate nerve activity. The invention also relates to methods of manufacture the compositions.

### BACKGROUND OF INVENTION

Pain has several different classifications including those based on origin, and those based on duration. The origin of pain may for example be nociceptive or neuropathic. Nociceptive pain is a type of pain caused by damage to body tissue, which can be caused by external injury or a condition. Neuropathic pain is caused by damage or dysfunction of the nervous system. The duration of pain may be classified as acute or chronic. Acute pain comes on quickly but lasts a short time (typically less than three months). Chronic pain is persistent and long-lasting.

In all cases the sensation of pain at some level is caused by relaying of electrical signals along different pain sensing neurons to the brain where the signal is interpreted. It follows that suppression of activity of these pain sensing neurons should result in pain relief.

An important condition featuring many different types of pain is osteoarthritis. Osteoarthritis (OA) is an incurable complex disease impacting 350 million people worldwide, including 40 million in Europe. OA is a degenerative joint disease where progressive degradation of bone and cartilage in the affected joint results in severe, typically chronic pain, reduced joint function, and stiffness. Overtime the pain, reduced function, and stiffness gets increasingly worse leading to ever greater patient debilitation.

Osteoarthritis most commonly affects the knee joint, representing 45% of cases (18 million Europeans). Knee OA is the 4th leading cause of disability worldwide, with rapid increasing prevalence due to an ageing population and the growing obesity epidemic. Knee osteoarthritis affects more people globally each year than cancer and heart disease. There is no means to stop disease progression, and no long-lasting effective pain relief treatment. Osteoarthritis pain is particularly severe impacting patients' sleep, mood, and everyday life. Patients with osteoarthritis suffer from chronic pain for an average of 28 years from initial diagnosis, as well as suffering immobility. Inadequate management of chronic pain is directly related to quality of life with one third of people with osteoarthritis over 45 suffering with high levels of depression and sleep deprivation.

Knee osteoarthritis is a painful, debilitating, and progressive disease where no current treatment offers patients effective pain relief without side effects. Current treatment options for knee osteoarthritis patients fall into four categories, with the patient pathway typically progressing through these options with disease progression:
Oral Medications: These include non-steroidal anti-inflammatory drugs (NSAIDs) and opioids. Both these classes of drugs manage the symptoms but are short acting usually requiring daily dosing and have serious side-effect such as organ damage with NSAIDs and dependency with opioids. Furthermore, as no agent stops progression of osteoarthritis, stronger doses are needed as the disease progresses and naturally there is a limit on the safety of increasing the oral doses. Steroid Injectables: Steroids are injected directly into the intra-articular space of the joint to give pain relief. However, the effect lasts for an average of only 2-4 weeks and irreplaceable cartilage is destroyed each time steroids are used leading to limitations on use.
Hyaluronic Acid (HA) Injectables: HA is a major component of the fluid in a normal healthy joint, acting as a lubricant and shock absorber. Commercial HA injectables replace HA in the damaged joint and while safe, only provide mild pain relief through a barrier effect. This lack of efficacy has resulted in bodies such as The American Academy of Orthopaedic Surgeons (AAOS) and NICE (UK) no longer recommending HA as a treatment for osteoarthritis.
Stem Cell and Platelet Injectables: Although currently an area of active research, these products have failed do demonstrate that they are effective in relieving OA pain.
Knee Replacement Surgery: Approximately 5.6% of patients every year opt for knee replacement surgery. This is an end-stage treatment where the damaged knee is replaced with an artificial metal implant. This is an expensive highly invasive procedure, that is often associated with a high failure rate and need for revision. Hence, surgery is considered a last-resort treatment for patients with terminally damaged knee joints and not responding to other treatment options.

Of relevance and note are the challenges in managing acute pain post-knee replacement surgery. Knee replacement surgery is associated with severe acute pain after surgery, and a high incidence of chronic pain. Currently a multimodal approach to treating the acute pain involving opioids, NSAIDs, and local anaesthetic nerve blocks is often used. Despite these measures to manage the acute pain, many patients are discharged after surgery with pain that can persist for weeks, months, or years with this chronic pain difficult to treat.

It is noteworthy that the current established treatments do not include an option primarily targeting modulation of afferent pain nerve activity. Suppression of inappropriate pain sensing nerve activity would be of clear benefit in osteoarthritis where there is such pain.

The inventors have introduced a peptide-based treatment for locally treating osteoarthritis in PCT/IB2022/061042.

In addition to application in OA conditions (including post-knee replacement surgery), it is recognised that agents that can be used to modulate the activity of pain sensing neurons may also be useful for other painful conditions such as those featuring neuropathic pain and/or inflammatory pain. These conditions include painful bladder syndrome, vaginal atrophy, and many others.

Further, in all cases it would be desirable that a therapy offers immediate, long-lasting, and safe modulation of nerve cell activity, and only in the specific nerve cell type—for example pain sensing neurons.

In addressing the above-mentioned issues, the inventors here focused on pharmacologically active agents selectively targeting pain sensing neurons. In particular, the inventors explored the modulation of ion channels in the membranes of peripheral pain sensing neurons such as voltage gated sodium (Nav) and the transient receptor potential cation channel subfamily V member 1 (TRPV1) channels by ion channel modulators. These channels are critical to the transmission of pain signals to the brain, with suppression of their overactivity leading to pain relief.

The use of ion channel modulators has been reported in certain limited conditions: The classic local anaesthetic class of drugs (e.g., lidocaine) act on the intracellular part of the Nav channel as ion channel modulators. While effective at pain suppression local anaesthetics typically suffer from low potency, short half-life, poor bioavailability, and lack of selectivity with associated adverse effects. These factors limit their utility in cases where there is a need for sustained pain relief. Hence, these agents have some limited topical uses but are more often given by injection for indications where there is a need for transient pain suppression, for example before surgical procedures. Attempts have been made to prolong their duration of action using technologies such as liposomes extending effects from hours to 2-3 days. However, the inherent low potency of these agents results in the need for large volumes to realise this increase in duration of action. For example, bupivacaine is usually administered at a strength of 0.25-0.75% (i.e., 25 mg-75 mg in a 10 ml volume), effect lasting for approximately 6 hours. The long-acting liposomal product Exparel® has a maximal dosage of 266 mg/20 ml with the SPC often indicating a recommendation to dilute depending on the indication. Exparel® has a duration of action of up to 72 hours. It is noted that intra-articular injection volumes into the knee as an example are of the order of 2-6 ml volume. In addition, the ubiquitous presence of Nav channels on many different nerve types and other excitable tissues can lead to side effects. For example, while local anaesthetics preferentially act on small fibre nerves such as those transmitting pain, they can also suppress the activity of larger nerves such as those involved in proprioception and motor function. While administration to the knee joint in the case of knee OA should limit systemic adverse effects a further limitation to use here is the chondrotoxic potential of these agents. Consequently, there is a need for compositions and methods to treat conditions such as OA, that provide long lasting effects, and that allow administration of higher concentrations of anaesthetics in low volumes.

So called Site 1 Nav antagonists such as tetrodotoxin are ultra-potent and could in theory be formulated as a depot giving sustained pain-relieving effect for conditions such as knee OA. However, they have a lack of selectivity and a very narrow therapeutic window limiting their therapeutic potential to date.

The TRPV1 channel is found mainly on small to medium fibres—i.e., mainly on pain sensing fibres. Importantly, these channels are upregulated and sensitised in pain conditions. These features make them an attractive target for candidate pain relieving drugs. However, attempts to target TRPV1 to date has met with limited success. Candidate TRPV1 antagonists (a type of ion channel modulator) for example have persistent issues with side effects such as hyperthermia and alteration of heat sensation. TRPV1 agonists such as capsaicin and resiniferatoxin paradoxically show more translation potential. Exposure to these TRPV1 agonists leads to initial activation (and pain perception) but then long-term desensitisation after sustained or high dose exposure.

While promising, the acute pain sensitisation requires significant levels of pre-treatment to control. In addition, the effect may begin to wane towards the end of the dosing interval leading to breakthrough pain before the next application.

Hence there is a need for new treatments for pain in conditions such as knee OA which are efficacious, safe, and long-lasting. Ion channel modulators (including some neurotoxins) acting on pain sensing neurons hold promise for use in these conditions but have challenges in their current form.

This invention provides solutions to the above-mentioned issues of the current art.

### SUMMARY OF THE INVENTION

The core concept of this invention is the conjugation and optionally additionally mixing of ion channel modulators to a biocompatible polymer base to overcome the above-described challenges. The conjugation and the optional mixing results in a depot of ion channel modulator which would be released over time in-vivo resulting in a profile within the therapeutic range and with an extended duration of action.

According to one aspect of the present invention, there is provided a composition comprising a polymer in a carrier and
i. At least one ion channel modulator covalently bound to the polymer; and optionally additionally
ii. At least one ion channel modulator, mixed with the polymer.

According to another aspect of the present invention, there is provided a method of localised modulation of nerve cell activity in a subject, the method comprising locally administering an ion channel modulator comprising a polymer in a carrier and
i. At least one ion channel modulator covalently bound to the polymer; and optionally additionally
ii. At least one ion channel modulator, mixed with the polymer.

Advantageously, the present invention provides ion channel modulators, that are covalently bound to the polymer base (which may be termed herein as “RM-010”), and are capable of modulating ion flow into nerve cells and to reduce the ability of these cells to generate action potentials. This reduction in action potential translates into pain relief when applied to pain sensing neurons (which has been demonstrated both in-vitro and in-vivo).

Furthermore, in the case of ion channel modulators of the TRPV1 agonist class when covalently bound to and optionally additionally mixed with a polymer base does modulate ion flow into nerve cells and reduce the ability of cells to generate action potentials and reduce the initial excitation phase characteristic of these modulators. This reduction in action potentials with a reduction in initial excitation translates into pain relief with a reduced initial acute period of sensitisation when applied to pain sensing neurons (which is demonstrated both in-vitro and in-vivo).

The present invention provides a solution, where covalent binding and optionally additionally mixing of ion channel modulators to the polymer provides a long-lasting effect when used for the modulation of nerve cells to provide for example pain relief. Moreover, the present invention provides a solution to reduce toxicity and safety signals related to the ion channel modulator.

Accordingly, it is an object of this invention to provide a composition for treatment of pain, wherein the composition comprises a carrier, and a polymer covalently linked to at least one class of ion channel modulators selected from the group consisting of modulators of transient receptor potential cation channel subfamily V member 1 (TRPV1) channels, voltage gated sodium (Nav) channels, voltage gated calcium (Cav) channels, and combinations thereof; and optionally additionally at least one class of ion channel modulators being mixed with the polymer.

According to certain embodiments the composition comprises one or more TRPV1 modulators selected from the group consisting of capsaicin (e.g. (Z)-capsaicin or (E)-capsaicin), capsaicinoids, capsinoids resiniferatoxin (RTX), olvanil, C18-Nacetylethanomimes, MSP-3 and vanilotoxin.

According to certain embodiment the composition comprises one or more Nav modulators selected from the group consisting of lidocaine, bupivacaine, ropivacaine, tetracaine, and tetrodotoxin.

According to some embodiments the composition comprises one or more Cav modulators selected from ziconotide and gabapentinoids including gabapentin and pregabalin.

According to certain embodiment the polymer is a biocompatible polymer and is selected from the group consisting of poly-ethylene glycol (PEG), poly(lactic-co-glycolic acid) (PLGA), polylactic acid, polysaccharides, silica, hyaluronic acid (HA) or an ester or salt thereof, polycaprolactone (PCL), polyamides and poly(ester-amide)s, polyurethanes, polyanhydrides, polyvinyl alcohol, chitin, cellulose, alginic acid or alginate, poly(hydroxybutyrate) (PHB), starch and its modified polymers, dendrimers, collagen, gelatin, calcium hydroxyapatite, polyethylene glycol diacrylate, sodium hyaluronate, polyglycolide, poly-3-hydroxybutyrate, and chitosan; or combinations thereof. The polymer may be cross linked.

According to certain embodiments the polymer may be HA or PEG or combinations thereof, and molecular weight of the polymer is between 50 and 3000 kDa.

According to certain embodiments the ion channel modulator directly or via a linker selected from the group consisting of beta-alanine, glycine, 5-aminovaleric acid; (8-amino-3,6-dioxaoctanoic acid); and (12-amino-4,7,10-trioxadodecanoic acid). The covalent link between the polymer and ion channel modulators may be selected from the group consisting of ester bonds, amide bonds, carbamate bonds, carbonate bonds, and urethane bonds.

According to certain embodiment the polymer is covalently linked to the ion channel modulator in a in a mass:mass ratio in a range of 1:0.001 to 1:5 polymer to ion channel modulator.

It is an object of the invention to provide a composition for pain relieve comprising a carrier; a polymer selected from the group consisting of HA, PEG, and a combination thereof; at least one TRPV1 modulator covalently linked to the polymer in a mass:mass ratio of 1:0.03, and the composition has a sustained release of the ion channel modulator. According to certain embodiments the TRPV 1 modulator may be capsaicin or resiniferatoxin, and the polymer hyaluronic acid having molecular weight between 500 and 1000 kDa.

According to certain embodiments the composition is suitable for administration as an injection.

It is an object of this invention to provide a method to release at least one ion channel modulator in a sustained and prolonged manner at a location of pain, said method comprising injecting the composition at the location of pain.

According to certain embodiments an injection volume may be 1-10 ml and the injection volume may comprise from 1 μg to 150 mg of ion channel modulator per millilitre.

### The Polymer

The polymer may be natural or synthetic. The polymer may be biocompatible. In a preferred embodiment, the polymer is a biodegradable polymer. Preferable polymers are non-toxic to mammalian subjects at therapeutically acceptable levels.

In one embodiment, the polymer is selected from one or more of poly-ethylene glycol (PEG), poly(lactic-co-glycolic acid) (PLGA), polylactic acid, polysaccharides, silica, hyaluronic acid or an ester or salt thereof, polycaprolactone (PCL), polyamides and poly(ester-amide)s, polyurethanes, polyanhydrides, polyvinyl alcohol, chitin, cellulose, alginic acid or alginate, poly(hydroxybutyrate) (PHB), starch and its modified polymers, dendrimers, collagen, gelatin, calcium hydroxyapatite, polyethylene glycol diacrylate, sodium hyaluronate, polyglycolide, poly-3-hydroxybutyrate, and chitosan; or combinations thereof.

The polymer may comprise or consist of glycosaminoglycans. In one embodiment, the polymer comprises, or consists of, hyaluronic acid (HA) or sodium hyaluronate. Hyaluronic acid (HA) may also be known as hyaluronan.

In another embodiment the polymer is a derivative of HA, such as a cross-linked HA, or HA with functional groups designed to favourably adjust the physicochemical properties or facilitate drug loading.

In another embodiment the polymer comprises polyethylene glycol (PEG).

The polymer may comprise or consist of a polymer selected from any of the group comprising polyacrylamide; pectin; alginate; carboxymethylcellulose; methylcellulose; PLGA; PEG; polysaccharide, such as starch, cellulose, chitin, alginate, hyaluronate; proteins such as collagen, gelatin, casein, albumin; polyvinyl alcohol (PVA); polyvinylpyrrolidone (PVP); polyethyleneglycol (PEG); polylactic acid; and polyhydroxy acid (PHA), or combinations thereof. The polymer may comprise or consist of a polymer selected from any of the group comprising poly ([alpha]-hydroxyacids) including poly-lactide-co-glycolide (PLGA), poly-lactic acid, polyethyleneimine (PEI), polylactic or polyglycolic acids, poly-lactide poly-glycolide copolymers, and poly-lactide poly-glycolide polyethylene glycol copolymers, polyethylene glycol (PEG), polyesters, poly ([epsilon]-caprolactone), poly (3-hydroxy-butyrate), poly (s-caproic acid), poly (p-dioxanone), poly (propylene fumarate), poly (orthoesters), polyol/diketene acetals addition polymers, polyanhydrides, poly (sebacic anhydride) (PSA), poly (carboxybiscarboxyphenoxyphosphazene) (PCPP), poly [bis(p-carboxyphenoxy) methane](PCPM), poly (amino acids), poly (pseudo amino acids), polyphosphazenes, derivatives of poly [(dichloro) phosphazene], poly [(organo) phosphazenes], polyphosphates, polyethylene glycol polypropylene block co-polymers for example that sold under the trade mark Pluronics™, natural or synthetic polymers such as silk, elastin, chitin, chitosan, fibrin, fibrinogen, polysaccharides (including pectins), alginates, collagen, peptides, polypeptides or proteins, copolymers prepared from the monomers of any of these polymers, random blends of these polymers, any suitable polymer and mixtures or combinations thereof.

In another embodiment, the polymer may comprise or consist of a carbohydrate polymer such as dextrans, cyclodextrins, xanthan, alginate, gellan, pullulan, mannan, chitin, chitosan, agarose, or guar.

The polymer may have a molecular weight sufficient to form a gel under normal environmental conditions, for example at ambient room temperature (i.e., about 24° C.). In another embodiment, the polymer may have a molecular weight sufficient to form a solution or a colloid with a carrier (for example an aqueous carrier) under normal environmental conditions, for example at room temperature (i.e., about 24° C.). The polymer may be at a sufficient concentration and have a sufficient molecular weight to form a solution or a colloid or a gel with the carrier, under normal environmental conditions for example at room temperature (i.e., about 24° C.).

The polymer may have a molecular weight of at least 5 kDa. Alternatively, the polymer may have a molecular weight of at least 10 kDa. Further alternatively, the polymer may have a molecular weight of at least 20 kDa. Alternatively, the polymer may have a molecular weight of at least 50 kDa. The polymer may have a molecular weight of up to 3000 kDa. Alternatively, the polymer may have a molecular weight of up to 2200 kDa. In one embodiment, the polymer has a molecular weight of between about 5 kDa and about 3000 kDa. In another embodiment, the polymer has a molecular weight of between about 10 kDa and about 3000 kDa. In another embodiment, the polymer has a molecular weight of between about 20 kDa and about 3000 kDa. In another embodiment, the polymer has a molecular weight of between about 50 kDa and about 2200 kDa. In one embodiment, the polymer has a molecular weight of between about 50 kDa and about 3000 kDa. In an alternative embodiment, the polymer has a molecular weight of between about 50 kDa and about 2500 kDa. In an alternative embodiment, the polymer has a molecular weight of between about 50 kDa and about 2400 kDa. In an alternative embodiment, the polymer has a molecular weight of between about 500 kDa and about 2400 kDa. In one embodiment, the polymer has a molecular weight of between 2.2 MDa and 2.4 MDa.

In an embodiment where the polymer is HA, the HA may have a molecular weight of at least 5 kDa. Alternatively, the HA may have a molecular weight of at least 10 kDa. Further alternatively, the HA may have a molecular weight of at least 20 kDa. Alternatively, the HA may have a molecular weight of at least 50 kDa. The HA may have a molecular weight of up to 3000 kDa. Alternatively, the HA may have a molecular weight of up to 2200 kDa. In one embodiment, the HA has a molecular weight of between about 5 kDa and about 3000 kDa. In another embodiment, the HA has a molecular weight of between about 10 kDa and about 3000 kDa. In another embodiment, the HA has a molecular weight of between about 20 kDa and about 3000 kDa. In another embodiment, the HA has a molecular weight of between about 50 kDa and about 2200 kDa. In one embodiment, the HA has a molecular weight of between about 50 kDa and about 3000 kDa. In an alternative embodiment, the HA has a molecular weight of between about 50 kDa and about 2500 kDa. In an alternative embodiment, the HA has a molecular weight of between about 50 kDa and about 2400 kDa. In an alternative embodiment, the HA has a molecular weight of between about 500 kDa and about 2400 kDa. In one embodiment, the HA has a molecular weight of about 750 kDa. In one embodiment, the HA has a molecular weight of between 2.2 MDa and 2.4 MDa.

In an embodiment where the polymer is PEG, the PEG may have a molecular weight of at least 5 kDa. Alternatively, the PEG may have a molecular weight of at least 10 kDa. Further alternatively, the PEG may have a molecular weight of at least 20 kDa. Alternatively, the PEG may have a molecular weight of at least 50 kDa. The PEG may have a molecular weight of up to 3000 kDa. Alternatively, the PEG may have a molecular weight of up to 2200 kDa. In one embodiment, the PEG has a molecular weight of between about 5 kDa and about 3000 kDa. In another embodiment, the PEG has a molecular weight of between about 10 kDa and about 3000 kDa. In another embodiment, the PEG has a molecular weight of between about 20 kDa and about 3000 kDa. In another embodiment, the PEG has a molecular weight of between about 50 kDa and about 2200 kDa. In one embodiment, the PEG has a molecular weight of between about 50 kDa and about 3000 kDa. In an alternative embodiment, the PEG has a molecular weight of between about 50 kDa and about 2500 kDa. In an alternative embodiment, the PEG has a molecular weight of between about 50 kDa and about 2400 kDa. In an alternative embodiment, the PEG has a molecular weight of between about 500 kDa and about 2400 kDa. In one embodiment, the PEG has a molecular weight of about 750 kDa. In one embodiment, the PEG has a molecular weight of between 2.2 MDa and 2.4 MDa.

The polymer may be cross-linked, for example to form a gel, or in another embodiment, the polymer may not require cross-linking. The polymer cross-linking may be covalent cross-linking or ionic or a blend of both, for example via charged functional moieties bound to the polymer.

The polymer, such as hyaluronic acid, may be provided in a concentration of up to 30% w/v in the carrier. The polymer, such as hyaluronic acid or PEG, may be provided in a concentration of between 0.1 mg/ml and 300 mg/ml. In another embodiment, the polymer, such as hyaluronic acid or PEG, may be provided in a concentration of between 5 mg/ml and 20 mg/ml.

### Covalent Attachment/Linked

In one embodiment the ion channel modulator(s) are covalently linked to the polymer by an ester bond. In another embodiment the ion channel modulator(s) are covalently linked to the polymer by an amide bond. The skilled person will be familiar with methods of covalently linking the ion channel modulator to the polymer.

The ion channel modulator(s) may be covalently linked to the polymer, such as HA or PEG, through covalent linkage using direct linkage via for example an ester or amide bond.

Alternatively, the ion channel modulator(s) may be covalently linked to the polymer by chemical bonding via a linker. The linker may comprise a chemical bond between atoms, such as C—C, C═C, C(O)—NH, C(N)═O, C—O, C—S, C(O)═O, C—N, C—S, or S—S.

Advantageously, these bonds (direct or via linker for example) can be characterised by different stability under different environmental conditions. Thus, by inclusion of chemically labile bonds (such as a disulphide bond) one can engineer bioresponsive materials that can be disassembled upon certain conditions (as for example 1000-fold higher concentration of thiols in cellular cytoplasm as compared with extracellular environment).

The linker molecule may be an organic molecule. The linker molecule may have one or more of the functional groups from any one of acrylamide, alkane, alkene, alkyne, alcohol, aldehyde, ketone, carboxylic acid, amino acid (or oligopeptide), ester, cycloalkane, sugar, and nucleic acid. One preferred linker molecule is a beta-alanine linker. The linker molecules may be of different lengths, exemplary wise the linkers may be selected from glycine, 5-aminovaleric acid (5 bonds long); (8-amino-3,6-dioxaoctanoic acid) (9 bonds long); (12-amino-4,7,10-trioxadodecanoic acid) (13 bonds long), other linkers may also be used.

The linker molecule may be arranged to be anchored to the polymer by chemical reaction. The chemical reaction may be spontaneous (i.e. self-catalysed), chemically catalysed or photo-catalysed. The linker molecule may comprise a thiol moiety for linking the linker molecule to the polymer by chemical reaction. The linker molecule may comprise a carboxylic acid, amide, alcohol, or other reactive moiety for linking the linker molecule to the polymer by chemical reaction. The linker molecule may comprise a disulphide linked pyridine for linking the linker molecule to the polymer by chemical reaction. The polymer may comprise an alcohol group, carboxylic acid group, amide group or other reactive moiety such as a disulphide linked pyridine, or a thiol moiety for linking the polymer to the linker molecule by chemical reaction.

Another part of the linker may then be anchored to the ion channel modulator by chemical reaction. The chemical reaction may be spontaneous (i.e. self-catalysed), chemically catalysed or photo-catalysed. The linker molecule may comprise a thiol moiety for linking the linker molecule to the ion channel modulator by chemical reaction. The linker molecule may comprise a carboxylic acid, amide, alcohol, or other reactive moiety for linking the linker molecule to the ion channel modulator by chemical reaction. The linker molecule may comprise a disulphide linked pyridine for linking the linker molecule to the ion channel modulator by chemical reaction. The ion channel modulator may comprise an alcohol group, carboxylic acid group, amide group or other reactive moiety such as a disulphide linked pyridine, or a thiol moiety for linking the ion channel modulator to the linker molecule by chemical reaction.

A benefit of covalently linking and optionally additionally mixing the ion channel modulator(s) to the polymer is that it can remain in situ, such as in a joint, for longer, giving long lasting effects such as pain relief. The ion channel modulator without the polymer is more quickly broken down and removed from the joint. Advantageously, the linking and optionally additionally mixing of the ion channel modulator(s) to the polymer can provide a therapy that has a clinically acceptable injection frequency, such as 2-4 times per year. The polymer linking may reduce degradation and prevent escape of the ion channel modulator(s) by trans-synovial flux in a joint. A HA polymer backbone may be protected from hyaluronidase degradation and mechanical degradation from compression. The specific distribution of the ion channel modulator(s) can avoid non-targeted biodistribution. Furthermore, the ion channel modulator(s) may provide the benefit of charge maintenance, where it stops electrostatic charge loss in the joint.

Furthermore, higher doses of ion channel modulator(s) may have safety issues as the dose moves outside the usual therapeutic window. Advantageously, the linking or mixing of higher doses of ion channel modulator(s), to the polymer can provide a therapy that is safe where the toxic properties of the ion channel modulator have been removed by virtue of conjugation.

### The Ion-Channel Modulator

The ion-channel modulator is understood to mean a molecule which interacts pharmacologically with ion-channel(s) on excitable tissues, in particular on nerves.

The ion-channel modulator may target voltage gated sodium (Nav) channels. In one embodiment the ion-channel modulator is a non-specific Nav antagonist. In another embodiment the ion-channel modulator is a specific Nav antagonist of one (or more) of the recognised members of the Nav channel family, such as Nav 1.7 or 1.8. In another embodiment the ion-channel modulator is an antagonist for specific members of the Nav channel family but with varying affinities. In another embodiment the ion-channel modulator is a mixed agonist/antagonist for the Nav channel (non-specific, specific, or mixed affinities). In another embodiment the ion-channel modulator is an agonist for the Nav channel (non-specific, specific, or mixed). In one embodiment the ion-channel modulator interacts with the Nav channel with reversible binding. In another embodiment the ion-channel modulator interacts with the Nav channel with irreversible binding. In one embodiment the ion-channel modulator interacts with the Nav channel through an extracellular domain. In another embodiment the ion-channel modulator interacts with the Nav channel through an intracellular domain. According to certain embodiments the ion-channel modulator is selected from lidocaine, bupivacaine, ropivacaine, tetracaine, and tetrodotoxin.

Alternatively, the ion channel modulator may target voltage gated calcium (Cav) channels. In one embodiment the ion-channel modulator is a non-specific Cav antagonist. According to certain embodiments the ion-channel modulator is selected form ziconotide and gabapentinoids (such as gabapentin and pregabalin). In another embodiment the ion-channel modulator is a specific Cav antagonist of one (or more) of the recognised members of the Cav channel family, such as N-type Cav channels. In another embodiment the ion-channel modulator is an antagonist for specific members of the Cav channel family but with varying affinities. In another embodiment the ion-channel modulator is a mixed agonist/antagonist for the Cav channel (non-specific, specific, or mixed affinities). In another embodiment the ion-channel modulator is an agonist for the Cav channel (non-specific, specific, or mixed). In one embodiment the ion-channel modulator interacts with the Cav channel with reversible binding. In another embodiment the ion-channel modulator interacts with the Nav channel with irreversible binding. In one embodiment the ion-channel modulator interacts with the Cav channel through an extracellular domain. In another embodiment the ion-channel modulator interacts with the Nav channel through an intracellular domain.

Alternatively, the ion channel modulator may target transient receptor potential cation channel subfamily V (TRPV) channels. In one embodiment the ion-channel modulator is a non-specific TRPV antagonist. In another embodiment the ion-channel modulator is a specific TRPV antagonist of one (or more) of the recognised members of the TRPV channel family, such as TRPV1 channels. In another embodiment the ion-channel modulator is an antagonist for specific members of the TRPV1 channel family but with varying affinities. In another embodiment the ion-channel modulator is a mixed agonist/antagonist for the TRPV1 channel (non-specific, specific, or mixed affinities). In another embodiment the ion-channel modulator is an agonist for the TRPV1 channel (non-specific, specific, or mixed). In one embodiment the ion-channel modulator interacts with the TRPV1 channel with reversible binding. In another embodiment the ion-channel modulator interacts with the TRPV1 channel with irreversible binding. In one embodiment the ion-channel modulator interacts with the TRPV1 channel through an extracellular domain. In another embodiment the ion-channel modulator interacts with the TRPV1 channel through an intracellular domain.

In one embodiment the ion channel modulator is the TRPV1 agonist capsaicin (e.g. (Z)-capsaicin or (E)-capsaicin), or a related capsaicinoids or capsinoids. In another embodiment the ion channel modulator is the TRPV1 agonist resiniferatoxin (RTX). In further embodiments the TRPV1 agonist is selected from olvanil, C18-Nacetylethanomimes, MSP-3 and vanilotoxin.

Alternatively, the composition may comprise a range of different ion-channel modulators, each targeting different ion channels and/or with different patterns of binding. In addition, the proportion of different ion-channel modulators can vary. In one embodiment, Nav channel modulators comprise the majority. In one embodiment, Cav channel modulators comprise the majority. In one embodiment, the TRPV channel modulators comprise the majority.

In one embodiment the composition may comprise a polymer that is one polymer of one mass. In another embodiment the polymer used has a range of masses. In another embodiment a blend of polymers is used of the same mass or as a range of masses.

In one embodiment the composition has a polymer component of weight of between 500 kDa-100 MDa. In one embodiment the composition has a polymer component of weight of between 2.2 and 2.4 MDa. In another embodiment the composition has a polymer component of weight of approximately 500 Da to 2000 kDa, and up to maximum size of approximately 100 MDa. In one embodiment the polymer is hyaluronic acid. In another embodiment the polymer is another biocompatible polymer. In another embodiment the polymer is a blend of different biocompatible polymers.

In one embodiment the composition comprises or consists of hyaluronic acid covalently linked to capsaicin, resiniferatoxin, or combinations thereof. In another embodiment the composition comprises capsaicin, resiniferatoxin; or combinations thereof covalently linked and optionally additionally mixed with hyaluronic acid. In one embodiment the composition comprises hyaluronic acid mixed (non-covalently bound) and covalently linked to capsaicin, resiniferatoxin; or combinations thereof. According to certain embodiments the composition may comprise or consist of PEG covalently bound to capsaicin, resiniferatoxin or their combinations. Because HA at lower component weights (<500 kDA) may have inflammatory effects, incorporating PEG allows the use of the lower component weight polymer without any inflammatory response.

In one embodiment the polymer is covalently linked to the ion channel modulator in a mass:mass ratio of about 1:0.2 polymer to ion channel modulator. In another embodiment the polymer is covalently linked to the ion channel modulator in a mass:mass ratio of about 1:0.03 polymer to ion channel modulator. In one embodiment the polymer comprises HA covalently linked to capsaicin in a mass:mass ratio of about 1:0.2 polymer to ion channel modulator. In another embodiment the polymer comprises HA covalently linked to resiniferatoxin in a mass:mass ratio of about 1:0.03 polymer to ion channel modulator. In another embodiment the polymer comprises or consists of HA covalently linked to the ion channel modulator, wherein the ion channel modulator comprises or consists of capsaicin or resiniferatoxin in a mass:mass ratio of between about 1:0.001 and about 1:5 polymer to ion channel modulator. In another embodiment the polymer(s) is/are covalently linked to the ion channel modulator(s) in a mass:mass ratio of between about 1:0.001 and about 1:5 polymer(s) to ion channel modulator(s).

In one embodiment the composition may additionally comprise the polymer mixed with the ion channel modulator in a mass:mass ratio of about 1:0.2 polymer to ion channel modulator. In another embodiment the polymer may be mixed with the ion channel modulator in a mass:mass ratio of about 1:0.03 polymer to ion channel modulator. In one embodiment the polymer comprises HA mixed with to capsaicin in a mass:mass ratio of about 1:0.2 polymer to ion channel modulator. In another embodiment the polymer comprises HA mixed with resiniferatoxin in a mass:mass ratio of about 1:0.03 polymer to ion channel modulator. In another embodiment the polymer comprises consists of HA mixed with the ion channel modulator, wherein the ion channel modulator comprises consists of capsaicin or resiniferatoxin in a mass:mass ratio of between about 1:0.001 and about 1:5 polymer to ion channel modulator. In another embodiment the composition may comprise polymer(s) mixed with ion channel modulator(s) in a mass:mass ratio of between about 1:0.001 and about 1:5 polymer(s) to ion channel modulator(s).

In another embodiment the polymer is mixed (non-covalently bound) and covalently linked with the ion channel modulator in a mass:mass ratio of about 1:0.2 polymer to ion channel modulator. In another embodiment the polymer is mixed (non-covalently bound) and covalently linked with the ion channel modulator in a mass:mass ratio of about 1:0.03 polymer to ion channel modulator. In one embodiment the polymer comprises HA mixed (non-covalently bound) and covalently linked to capsaicin in a mass:mass ratio of about 1:0.2 polymer to ion channel modulator. In another embodiment the polymer comprises HA mixed (non-covalently bound) and covalently linked with resiniferatoxin in a mass:mass ratio of about 1:0.03 polymer to ion channel modulator. In another embodiment the polymer comprises consists of HA mixed (non-covalently bound) and covalently linked with the ion channel modulator, wherein the ion channel modulator comprises capsaicin or resiniferatoxin in a mass:mass ratio of between about 1:0.001:0.001 and about 1:5:5 polymer to mixed (non-covalently bound) ion channel modulator to covalently linked ion channel modulator. In another embodiment the polymer(s) is/are mixed (non-covalently bound) and covalently linked with ion channel modulator(s) in a mass:mass:mass ratio of between about 1:0.001:0.001 and about 1:5:5 polymer(s) to mixed (non-covalently bound) ion channel modulator(s) to covalently linked ion channel modulator(s).

### Composition Properties

The composition may be in the form of a gel, colloid, suspension, or solution. In one embodiment, the composition is a gel, for example as formed by the polymer.

The composition may have a zeta-potential in the range of −50 mV to +50 mV. The gel may have a viscosity of between 10⁰ and 10⁶ mPa·s.

Advantageously, the provision of the composition in the form of a gel may provide the additional benefit of providing similar properties, such as viscosity and rheology to natural joint synovial fluid and HA, which can contribute to joint function, joint pain relief and sustained pain relief, where the molecule takes longer to be removed and broken down from the joint.

### The Carrier and Other Additives

The carrier may be a pharmaceutically acceptable carrier. The carrier may be a liquid carrier. The carrier may be fluid based for example as an aqueous, organic, or multiphasic liquid, or may be solid in nature. The carrier may be gel or a paste. In one embodiment, the carrier is an aqueous carrier. In one embodiment, the carrier is aqueous and comprises of water or saline or a buffer or buffered saline. Nonlimiting examples of the carrier are phosphate buffered saline PBS, and (2-(N-morpholino)ethanesulfonic acid) (MES). A person skilled in the art would be able to choose other buffers for use as carrier as well. Generally, a pH range of 4.0-7.4 is preferable. The carrier may further comprise a further active agent. The active agent may be a therapeutically, prophylactically or diagnostically active substance. The active agent may be a bioactive substance. The active agent may be selected from the group comprising a drug, pro-drug, peptide, protein, and nucleic acid, or combinations thereof. The active agent may comprise or consist of a biomolecule.

The composition may be formulated for administration by a pharmaceutically or cosmetically acceptable route such as by injection or by topical application. The composition may further comprise pharmaceutically acceptable excipients. The composition may further comprise excipient(s) selected from the group consisting of pharmaceutically acceptable salts, polysaccharides, peptides, proteins, amino acids, synthetic polymers, natural polymers, and surfactants.

### The Cells

In one embodiment, the composition is for localised modulation of nerve cell activity. Nerve cells may comprise cortical neurons, efferent neurons such as motor neurons, or afferent neurons such as pain-sensing nerves cells including dorsal root ganglion neurons (DRGs).

### The Subject

The subject may be a mammal. The subject may be human. The subject may have abnormal physiology/anatomy including tissue damage or tissue degeneration. The subject may have a bone fracture or degeneration. In one embodiment, the subject has osteoarthritis, for example of a joint, such as the knee, hip, shoulder, elbow or the joints between the various (meta)carpal/(meta)tarsal and phalangeal bones.

The subject may be suffering from increased neural activity selected from pain, hypertension, hyperhidrosis, and involuntary muscle spasms including dystonia. The subject may be suffering from spasticity, migraine, dystonia or essential tremor. The subject may be suffering from a pain, which is not neuropathic pain. The subject may be suffering from nociceptive pain. The subject may be suffering from a condition that requires modulation of excitable tissues such as neural and/or muscle cells including cardiac arrhythmias or skeletal muscle overactivity.

The subject may be an individual having knee replacement surgery or another orthopaedic surgery on a joint or bone. The subject may be an individual with post soft tissue surgery.

The subject may be suffering from pain from neuropathic pain, painful bladder syndrome, cystitis, or vaginal atrophy.

In another embodiment, the subject may have normal physiology/anatomy which it is desirable to modify, for example if undergoing or in need of cosmetic treatment.

### Administration

In one embodiment, the composition is, or is arranged to be, administered to subject by local injection, for example at the site of a pain, or infection, such as in a joint or tissue. The administration may be targeted to a specifically targeted nerve or muscle tissue. The target nerve may be an afferent nerve associated with a target site, the target site and associated afferent nerve being selected from:
Knee Joint—Genicular nerve or branches thereof; Hip Joint—Obturator nerve or branches thereof; Shoulder Joint—Suprascapular nerve or branches thereof (most common for shoulder pain); Elbow Joint—Radial nerve and Ulnar nerve or branches thereof; and Finger Joints—Superficial radial nerve; or combinations thereof.

The nerves may be located in any tissue. Any suitable nerve or nerve fibres may be targeted for pain relief and/or muscle/motor neuron inhibition and/or modulation of function. In one embodiment, the targeted nerve may be a peripheral nerve. The nerve may be a sensory neuron. In addition, any suitable muscle may be targeted for modulation of function, for example cardiomyocytes.

The composition may be administered by injection, with an injection volume of about 1-10 ml or 1-5 ml in humans, for example when administered to the intra-articular space of the knee joint. Additionally, or alternatively the composition may be administered at a dose in the range 1 mg/ml-30 mg/ml with respect to concentration of polymer. In one embodiment, the composition is administered with a dose of about 1 microg/ml to 150 mg/ml with respect to concentration of ion channel modulator(s).

According to another aspect of the invention, there is provided a method of manufacturing the composition according to the invention, the method comprising the step of covalently bonding ion channel modulator(s) to the polymer and forming a gel, colloid, or solution in a carrier.

According to another aspect of the present invention, there is provided a composition suitable in a method of treatment of a subject for pain or a motor neuron disorder selected from spasticity, migraine, dystonia or essential tremor, the method comprising the local administration of the composition to a subject at the site of the pain or motor neuron disorder, wherein the composition comprises a polymer in a carrier, wherein the polymer comprises one or more ion channel modulator(s) covalently bound to the polymer and optionally additionally ion channel modulator(s) mixed with the polymer.

The composition may be administered to the site of infection using local delivery systems including via injection, via instillation, or by topical routes. The composition may also be administered as a prophylactic treatment to prevent pain or other undesirable outcome from the inappropriate activity of excitable tissues; e.g., peri-surgical and post-surgical use.

In one embodiment, the pain is any pain associated with the peripheral nervous system (i.e., not the central nervous system). In one embodiment, the pain may a pain from neuropathic pain, nociceptive pain, painful bladder syndrome, cystitis, or vaginal atrophy.

The pain may be joint pain, such as from osteoarthritis, or tissue pain, for example from tissue damage, disease, or infection. The pain may typically be chronic pain but may also be acute pain.

The composition may be provided locally in a treatment, for example by injection at the site of the pain or motor neuron to be modulated. Migraine pain may be treated by localised injection into the forehead and/or temple of a subject.

According to another aspect of the present invention, there is provided the use of the composition according to the invention for pain relief or motor neuron modulation in a subject.

According to another aspect of the present invention, there is provided the use of the composition according to the invention for modulation of nerve cell activity, in a subject. The subject may be afflicted with a disorder associated with nerve cell activity.

According to another aspect of the present invention, there is provided the use of the composition according to the invention for treatment of conditions related to abnormal activity of muscle cells—for example cardiac arrhythmias by blockade of inappropriate pacemaker cell activity. The term “abnormal activity of muscle cells” may include an activity causing a pathology, such as irregular or excessive contraction of muscle cells, such as in arrhythmia.

The use may be for a cosmetic procedure, for example to relax facial skeletal muscles.

According to another aspect of the present invention, there is provided a kit comprising the composition according to the invention, and a syringe. The composition may be pre-loaded in the syringe. The kit may further comprise instructions for local administration of the composition to a site of pain, such as a joint, or a site for motor neuron blockade/modulation.

### Ion Channel Modulator Dose Treatment

According to an aspect of the invention, there is provided the use of the composition comprising one or more ion channel modulators, such as TRPV1 modulators, for the localised modulation of nerve cell activity, in a subject, wherein the treatment comprises the administration of the ion channel modulator(s) to the subject by localised injection into a tissue or joint at the site of the pain, and optionally wherein the ion channel modulator(s) are administered at a dose of 1-10 ml, 1-5 ml, 2-4 ml and about: 10 microg/ml to 100 mg/ml; 1 microg/ml to 150 mg/ml and 50 microg/ml to 20 mg/ml of ion channel modulator. The administration may be local administration.

The ion channel modulator may act on any one of the relevant ion channels on excitable tissues such as Nav, Cav, TRPV, or a combination thereof. These ion channel modulators are therapeutically effective at specific concentrations, however, at higher concentrations they exhibit safety signals including toxicity and other adverse events—for example acute pain in the case of TRPV1 agonists. Advantageously, the present invention establishes a concentration that is safe and effective for the therapeutic to modulate excitable cell activity without causing unacceptable adverse events. The conjugation results in a depot of ion channel modulator which is released over time in-vivo resulting in a profile within the therapeutic range, reducing the safety issues of bolus dosing, and with an extended duration of action.

### BRIEF DESCRIPTION OF THE DRAWINGS

FIG. 1A, B. Zeta-potential is measure of the effective electric charge on the nanoparticle's surface, quantifying the charges. The mean zeta potential of HA (1 mg/ml) was recorded at −23.2 mV and is shown in FIG. 1A. The mean zeta potential of the exemplary composition A (CL144-1) (1 mg/ml) was recorded at −30.4 mV and shown in FIG. 1B. Composition A is detailed in Table 2a.1. These results prove that composition A is highly anionic and when compared with Hyaluronic acid, the composition has a higher negative value. A zeta potential of range+/−60 mV indicates stability in the case of colloid systems. In the case of composition A, while conjugation with capsaicin may theoretically be expected to reduce the negative charge the opposite effect was seen to occur. This increased negative value compared to HA indicates compaction of the molecule with increased charge density.

FIG. 2: The ¹H-NMR spectrum of exemplary composition A (CL144-1) is shown. The peaks associated with HA are recorded between 3.3 ppm and 4.6 ppm and appear as broad peaks. Peaks associated with the aromatic protons of capsaicin are still distinctive and recorded between 6.6 ppm and 6.9 ppm. The peaks associated with the beta-alanine linker are also shown. As well as confirming structure, ¹H-NMR was also used to calculate the percentage conjugation of capsaicin—beta-alanine with HA. Composition A is detailed in Table 2a.1.

FIG. 3 illustrates the release kinetics of exemplary compositions A and B under different conditions (4° C., and 37° C. with agitation are shown). Compositions are prepared at 1 mg/ml and the levels of free capsaicin assayed using patch clamp at various timepoints up to 16 days. The results show the compositions to have good stability at 4° C. with no significant capsaicin release over a >7-day time-course. Both Compositions A and B show release of capsaicin over 12.5% of the conjugated load by day 3 at 37° C. with agitation, and over 50% by day 16. Composition A shows faster release kinetics compared to Composition B. The results demonstrate stability (4° C.) and favourable release kinetics under in-vivo conditions (37° C. with agitation). Compositions A and B are detailed in b).

FIG. 4 shows results for exemplary composition A (Table 2b.1). von Frey testing performed measuring paw withdrawal threshold (PWT) in naïve rodents with the area under the curve (AUC) calculated over time intervals post-treatment with intra-articular injection with respect to baseline. Results are shown at 24 h (left side set of columns) and at 2.3 h (right side set of columns). In each set of columns from left to right test materials are Vehicle, HA, CAP, CAP+HA, and exemplary composition A at three different concentration levels (x1, x5, x10 that of the CAP+HA concentration level). The results show exemplary composition A has a higher PWT compared to both capsaicin (CAP) and the mixture group (CAP+HA) demonstrating the benefit of conjugation of capsaicin in allowing loading of up to ×10 the levels of capsaicin without the same level of acute sensitisation (i.e. pain).

FIG. 5 shows in-vivo results for exemplary composition A (Table 2b.1). Duration of immobility measured following intra-articular administration of treatments saline control, capsaicin 0.2 mg/ml, and exemplary composition A at concentration level corresponding to conjugated capsaicin of 0.2 mg/ml in naïve rodents. The results show exemplary composition A leads to a duration of immobility (as a measure of pain) equivalent to that of control, and less than that of free capsaicin. This result demonstrates the benefit of conjugation of capsaicin in reducing the initial acute period of sensitisation.

### DETAILED DESCRIPTION OF THE INVENTION

### Definitions

A “gel” is understood to be a semi-solid matrix of polymers, which exhibits no flow when in the steady-state, although the continuous phase may still diffuse through this matrix.

The term “colloid” is understood to mean a dispersion of polymer and peptides in a carrier. The colloid is understood to be liquid in form.

The term “solution” is understood to mean the polymer and/or peptides are dissolved in a solvent carrier.

The term “biocompatible” is understood to include non-toxic to the human or animal body. To be biocompatible, the composition may not cause an immune response.

The term “biodegradable” is understood to include the ability to breakdown over time in the tissue or body of a human or animal, and/or in the environment. The time for complete degradation may be at least 1 week, at least 1 month, at least 2 months, at least 6 months, or at least 12 months. The time for complete degradation may be no more than 12 months. The time for complete degradation may be no more than 6 months.

The term “localised”, “local delivery”, “local administration” or “localised treatment” or similar, is intended to refer to the administration of treatment directly to a site of the pain or site of relevant pathology, for example by injection into the effected tissue, or directly into the afflicted joint. For delivery into a joint, the composition may be administered into a joint space, such as into the synovial fluid. The treatment may not be administered systemically.

A “covalent bond”, also called a molecular bond, is a chemical bond involving the sharing of electron pairs between atoms. These electron pairs are known as shared pairs or bonding pairs, and the stable balance of attractive and repulsive forces between atoms, when they share electrons, is known as covalent bonding.

The term “inhibition” used herein may be a total or partial inhibition of activity, such as channel activity or electrical activity.

The term “modulation” used herein may be a total or partial inhibition of activity or total or partial upregulation of activity or a change in the pattern of activity such as channel activity or electrical activity.

The skilled person will understand that optional features of one embodiment or aspect of the invention may be applicable, where appropriate, to other embodiments or aspects of the invention.

### DETAILED DESCRIPTION OF THE INVENTION

Embodiments of the invention will now be described in more detail in light of non-limiting experiments. The embodiments mentioned in this text relate, where applicable, to all aspects of the invention, even if this is not always separately mentioned.

This disclosure shows potential for a composition comprising a medically approved biodegradable polymer covalently bonded with ion channel modulators to provide a reduction of potential safety signals, efficacy in terms of desired neural modulation, and an elongated residency time for the ion channel modulator.

One aspect of the present disclosure comprises taking a medically approved biodegradable polymer, such as hyaluronic acid or a salt form thereof, chemically modified to have properties that modulate the activity of excitable cells such as neurons. One method to achieve this is to attach ion channel modulator(s) to polymer chains forming, or comprised in, the biodegradable polymer. One such group of ion channel modulators capable of modulating nerve activity are TRPV1 agonists such as capsaicin and resiniferatoxin. It is well reported in the literature that the mechanism in which this group of ion channel modulators work is by persistent activation of these receptors, resulting in initial excitation and then a long-term loss of response. The result is an initial increase in the sensation of pain (felt as warming, tingling, stinging, or burning) followed by a loss of pain sensation. Advantageously as TRPV1 receptors are more commonly found on smaller pain sensing fibres making the receptor a natural target for candidate analgesics. However, in the treatment of pain TRPV1 agonists suffer from the initial excitation often requiring concomitant analgesics such as opioids to mitigate against. Further, while long-lasting, the effect to TRPV1 agonists begins to wane after a time leading to the possibility of breakthrough pain.

The composition according to this invention contains one or more ion channel modulators either covalently bound and optionally additionally mixed with the biodegradable polymer. In clinical use the composition may be injected into the target area nearby the nerves of interest, where gradual and controlled release of the ion channel modulator(s) can be tailored in order to achieve:
Sustained exposure of the pain sensing neurons to ion channel modulators such as TRPV1 agonists, but at a level low enough not to illicit the initial excitation and pain.
Sustained release over a prolonged period to ensure therapeutic cover over the entire treatment interval reducing the risk of breakthrough pain.

Since the activation of voltage gated channels such as TRPV1 channels on pain sensing neurons is a necessary step for nerve conduction, the composition will reduce, or optionally effectively block, all pain signals from the nerve. Other channels exist that may act as a target and would give a similar overall effect on the nerve or excitable tissue, such as Nav and Cav channels. In one embodiment combination of different ion channel blockers may be used in the composition.

Chemical modification of the biodegradable polymer to attach ion channel modulator molecules can be achieved by the following methods:

The biodegradable polymer to which the ion channel modulator(s) are covalently linked are designed to ensure that the ion channel modulator(s) can detach in a controlled fashion and diffuse to their active site (i.e., ion channel of relevance) on the nerve. Suitable designs are based, for example, on the size of the polymer chains. The polymer chains of the biodegradable polymer can be designed to be any size from 10²-10²⁰ Da. Control to polymerisation chain lengths in polymer reactions is well documented in the scientific literature. For example, hyaluronic acid polymer chain length can be controlled by methods such as control of temperature with appropriately genetically engineered bacteria.

### Degradation of the Biodegradable Polymer In-Vivo

The biodegradable polymer can be designed to degrade fully in-vivo from 0.5 months to 5 years, more preferably from 5 months to 3 years, and most preferably from 6 months to one year. Degradation can be controlled and tailored to ensure the required degradation times are achieved. The main factors that determine the degradation process are the chemical structure of the polymer chain in the polymer backbone; hydrophilic/hydrophobic groups; polymer morphology; molecular weight; surface area; catalysts; and additives of resorbable polymers. The chemical structure of the polymer chain in the polymer backbone is the most important parameter. Generally, anhydrides degrade faster than the ester group, which in turn degrades faster than the amide group. Based on this, it is possible to predict degradation of a given polymer. The hydrophilic/hydrophobic character is another important parameter in polymer degradation. Poly(glycolic acid) degrades faster than the more hydrophobic poly(lactic acid), although they have the same degradable chain in the polymer backbone. The degradation rate is further influenced by polymer morphology. Crosslinkers may be used to slow down the degradation profile of hyaluronic acid. The crosslinkers bind HA (for example) polymer chains to each other both intra- and inter-molecularly, creating a polymer ‘network’. This network tends to be more stable, and physicochemically appears more viscous at a given concentration. This is used for example to transform a viscous liquid into a gel. This gel is a single unit, imposing a physical and chemical barrier to enzymatic and free radical breakdown. Because the gel network is connected at many multiple points, enzymes and free radicals can break down the chains only in much smaller portions at a time. Moreover, due to their large size, enzymes can have difficulty penetrating the gel network, which will in effect contribute to a slower degradation. This translates into longer persistence of the gel of the present invention at a target site in-vivo. Common crosslinkers include 1,4-butanediol diglycidal ether (BDDE), and di-vinyl sulfone (DVS). Both react with hydroxyl sites on the HA chains and offer similar results in slowing down enzymatic and free radical degradation of HA gels once injected. BDDE may be used as a crosslinker, binding together two HA polymer chains. BDDE (1,4-butanediol diglycidal ether) crosslinking agent may be used to bind HA polymer chains to each other, transforming liquid HA solutions into gels. Both the primary hydroxyl site (—CH₂OH) and secondary hydroxyl sites (—CHOH) within the HA monomeric unit are possible target sites for reaction with BDDE.

### Production of Hyaluronic Acid Gel

### a) Crosslinking of Hyaluronic Acid

Materials: Hyaluronic acid sodium salt (HA, also called hyaluronan or sodium hyaluronate) with an average molecular weight of 1.5×10⁶ Da, supplied by Shangdong Freda Biopharm Co., Ltd. (Jinan, China) as dry powders. 1,4-Butanediol diglycidyl ether (BDDE) was purchased from Sigma Chemical (St. Louis, MO, USA). Unless otherwise specified, all other chemical reagents used supplied by Sinopharm Chemical Reagent, of analytical grade and used as received without further purification.

Cross-linking of HA: HA first dissolved in 1% NaOH at a concentration of 10% w/w, after which BDDE was added to the HA solution with vigorous stirring. The final concentrations of BDDE were 0.4% v/v, 0.6% v/v, 0.8% v/v and 1% v/v, respectively. The solution was then allowed to crosslink at 40° C. for 5 h, followed by being dried at room temperature for 3 days. Phosphate buffered saline (PBS: NaCl, 9 mg·mL⁻¹; KH₂PO₄, 0.03 mg·mL⁻¹; Na₂HPO₄·2H₂O, 0.14 mg·mL⁻¹; pH 7.0) of 500 mL was then added to the above crosslinked HA to make it swell, after which it was put into dialysis bag and dialysed sequentially with excessive deionised (DI) water and PBS to remove the residual BDDE. The resulting gel was adjusted with PBS to obtain a gel with a HA concentration of 20 mg·mL⁻¹ and then used in a homogenizer to obtain gel particles of 0-400 nm in size. Before being used, the obtained injectable gel was sterilized in a high-pressure steam sterilizer set at 120° C., 20 min. When the biodegradable polymer is hyaluronic acid, the hyaluronic acid used for covalent linking with the ion channel modulator(s) can be used in a non-crosslinked form, or can be crosslinked, optionally as disclosed above.

The half-life of hyaluronic acid injected in-vivo non-crosslinked is of the order of hours-days, cross-linking has been shown to increase residence time to months using cross-linking agents like BDDE.

### b) Curing of the Gel In-Situ

The gel may be designed to be cured in-situ. This gives the added benefit to ensure the gel does not migrate from the target site and would also give the gel additional mechanical strength. It is difficult to inject very viscous materials; however, a low viscosity polymer solution could be injected to the target nerve site and by curing in situ the gel can cross-link and form a strong solid support of high mechanical strength. Curing in-situ would be achieved by using a chemical crosslinkers such as BDDE or divinyl sulfone at higher concentrations, promoting formation of a more solid polymer matrix in-vivo.

### c) Targeting the Gel to Different Nerve Fibres or Excitable Cells

The gel may be formulated to selectively target different nerve fibres or other excitable cells. This can be achieved by a concentration effect or by increasing the volume. For example, C-5 type pain fibres are non-myelinated and of 0.2-1.5-micron diameter while A-delta pain fibres are myelinated and of 1-5 micron diameter. These pain fibres are of smaller diameter to afferent or motor fibres. This differing anatomy offers the opportunity for a different pattern of ligands or gel to target pain fibres and not interfere with the other nerve types, thereby reducing potential adverse effects. For example, TRPV1 channels are selectively expressed on these small diameter fibres.

### d) Delivery of the Gel to Target Site

The gel may be delivered to the site of action (the vicinity of the target nerve/excitable tissue) using a needle attached to a syringe loaded with the gel, or by another system suited to localised delivery. A range of needles are available depending on the intended site of action, viscosity of the gel, and other factors. The injection volume can be dispensed in the usual fashion—i.e., navigation to site of administration and delivery of the gel as a bolus. Alternatively, to facilitate delivery of small amounts of gel in a relatively wide area encompassing the target nerve, the needle can have a mechanism that delivers aliquots of gel as a series of micro-injections. Initially, the clinician delivers the needle to a central area at which point a mechanism is engaged that causes the needle tip to partially retract/move/inject over a specified automated cycle to result in optimal widespread delivery of the gel.

The syringe could have a second chamber with a counteracting agent to ion channel modulator or another ligand attached to the polymer. This counteracting agent could be a direct antagonist of the ion channel modulator (for example, 4-aminopyridine may be used as an antidote to tetrodotoxin) or may cleave the group linking the ligand to the gel, thus de-anchoring the gel. Such a mechanism to counteract the ion channel modulator/nerve blocking ligand on the gel would be desirable in the event of excessive amount of the gel being delivered or incorrect initial placement. The counteracting agent could also be delivered in a separate syringe delivered by a similar or modified injecting pattern if there were unacceptable adverse effects experienced by the patient in the period after initial treatment.

Optionally, the counteracting agent may be one that causes direct break-up of the gel itself, for example, a chemical that lyses polymer cross-links. Such as the following: bacterial β-endoglycosidases, bacterial β-exoglycosidase, eukaryotic enzymes: β-Endoglycosidases, β-Exoglycosidases, hyaluronidases.

The skilled person will appreciate that different grades of gel with differing types and amount of ion channel modulator(s) can be designed for different target sites and for larger/smaller nerves. For example, the trigeminal nerve branches have quite distinct dermatomes meaning targeting of a given branch should allow targeting of effect to the respective dermatome. More extensive targeting may require delivery of ion channel modulator(s) containing gel to the trigeminal ganglion for widespread effect, or alternatively separate targeting of each branch.

A similar set of delivery systems could be employed where the target site is not suitable for administration with a needle. For example, a cannula/catheter for instillation into the bladder, or paints/lotions/creams/gels/sprays for topical delivery.

### Synthesis of Exemplary Compositions

### 0.1 Synthesis of Capsaicin-Hyaluronic Acid Using Ester Linkage

Hyaluronic Acid (HA) molecular weight approx. 0.75 MDa was purchased from Contripro (Czech Republic), capsaicin was purchased from Merck (Ireland). All other regents were purchased from Merck (Ireland) unless noted. A 1% w/w solution of HA (800 mg) was prepared in a 1:1 v/v (H₂O/DMSO) solvent mixture. To this mixture added DCC (N′-dicyclohexylcarbodiimide) (100 mg) and DMAP (4-(Dimethylamino)pyridine) (40 mg). Stirring of the solution was performed for a period of 1 hour in order to achieve activation of the carboxylic group of HA. 160 mg of capsaicin was dissolved in 50 ml of DMSO an slowly added to the above solution. The mixture is allowed react while stirring at 60-65° C. for about 6 hours—allowing an ester link be created between the HA —COOH groups and the phenol group of capsaicin. Other bond types, e.g., amide, carbonyl, ether, urethane may also be possible. The resultant solution is dialysed against DMSO and then against deionised water (3 days each) using a dialysis membrane (MWCO: 4000 Da) to remove impurities. The HA-capsaicin composition solution is then passed through a 0.22 μm filter to sterilise, then the product is lyophilised and then refrigerated at 4-8° C.

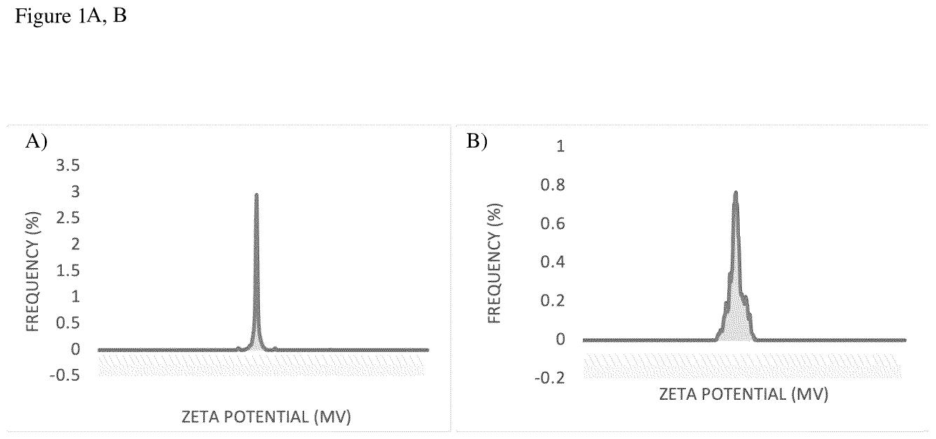

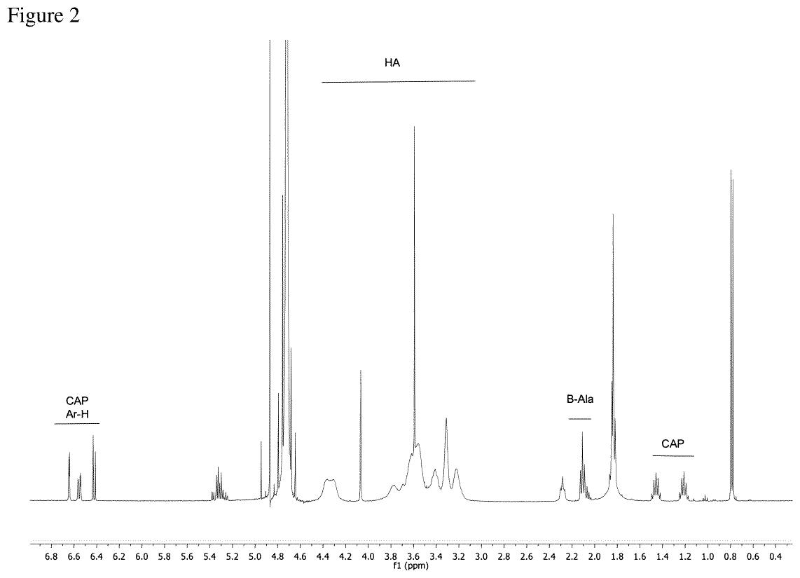

Characterisation: The structure is identified by ¹H-NMR (proton nuclear magnetic resonance spectroscopy) and mass spectrometry, with further characterisation performed using HPLC (high performance liquid chromatography) using to characterise sizing. These results will be confirmed using other modalities such as Fourier-transform infrared (FTIR) spectroscopy, and dynamic light scattering (DLS). FIGS. 1 A and B illustrate zeta-potential measurements of HA and exemplary composition A (see below). FIG. 2 illustrates the ¹H-NMR-spectroscopy results of exemplary composition A (see below).

### 0.2 Synthesis of Resiniferatoxin-Hyaluronic Acid Using Ester Linkage

A similar approach to that used in the example 0.1 above is to be used to link resiniferatoxin (RTX) with HA using ester linkages. 800 mg of HA is to be conjugated to 24 mg of RTX using the process described above.

### 0.3 Synthesis of Capsaicin-Hyaluronic Acid Using Amide Linkage Via Carbodiimide Coupling.

Hyaluronic Acid (HA) molecular weight approx. 0.75 MDa was purchased from Contripro (Czech Republic), capsaicin was purchased from Merck (Ireland). All other regents were purchased from Merck (Ireland) unless noted. A 1% w/w solution of HA (1000 mg) was prepared in MES (2-(N-morpholino)ethanesulfonic acid) buffer. The HA is activated using 1-ethyl-3-(3-dimethylaminopropyl)carbodiimide (EDC), at a molar ratio of 1:1.5 (HA:EDC). The reaction is carried out at pH 4.5-5.5 and room temperature for 30 minutes with gentle stirring. Capsaicin (1000 mg) is then dissolved in a small amount of DMSO (anhydrous dimethyl sulfoxide). In order to achieve conjugation, the activated HA solution is mixed with the activated capsaicin solution (1:5 mass ratio). To improve the reaction efficiency, N-hydroxysuccinimide (NHS) is added at a molar ratio of 1:1 with the reaction run at room temperature for 24 hours—allowing an amide link to be created between the HA —COOH groups and the secondary amine group of capsaicin. The resultant solution is purified using dialysis using DMSO and then deionised water (2 days each; dialysis membrane MWCO: 3000 Da). The HA-capsaicin composition solution is then passed through a 0.22 μm filter to sterilise, then the product is lyophilised and then refrigerated at 4-8° C.

Characterisation: The structure of the product, degree of conjugation and level of impurities are profiled using ¹H-NMR, mass spectrometry, with HPLC used to characterise sizing. These results are confirmed using other modalities such as FTIR spectroscopy, and DLS.

### 0.4 Synthesis of Capsaicin-Hyaluronic Acid Using Urethane Linkage.

Hyaluronic Acid (HA) molecular weight approx. 0.75 MDa) was purchased from Contripro (Czech Republic), capsaicin was purchased from Merck (Ireland). All other regents were purchased from Merck (Ireland) unless noted. A 1% w/w solution of HA (1000 mg) is prepared in MES (2-(N-morpholino)ethanesulfonic acid) buffer. The HA is then activated using N-(3-dimethylaminopropyl)-N′-ethylcarbodiimide hydrochloride (EDC), at a molar ratio of 1:1.5 (HA:EDC). The reaction is carried out at pH 4.5-5.5 and room temperature for 30 minutes with gentle stirring. Capsaicin (1000 mg) is then dissolved in a small amount of DMSO (anhydrous dimethyl sulfoxide). In order to achieve conjugation, the activated HA solution is mixed with the activated capsaicin solution (1:5 mass ratio). Next 1,6-hexamethylene diisocyanate (HDI) is added at a molar ratio of 1:1 to form urethane bonds between the two molecules. The reaction mixture is incubated at room temperature for 2-4 hours with gentle stirring.

The resultant solution is purified using dialysis using DMSO and then deionised water (2 days each; dialysis membrane MWCO: 3000 Da). The HA-capsaicin composition solution is then passed through a 0.22 μm filter to sterilise, then the product is lyophilised and then refrigerated at 4-8° C.

Characterisation: The structure of the product, degree of conjugation and level of impurities are profiled using ¹H-NMR, mass spectrometry, with HPLC used to characterise sizing. These results are confirmed using other modalities such as FTIR spectroscopy, and DLS.

### 0.5 Synthesis of Capsaicin-Hyaluronic Acid Compositions Using a Beta-Alanine Linker

Initially capsaicin—beta-alanine compositions are synthesised as follows:

To a solution of N-(tert-Butoxycarbonyl)-β-alanine (200 mg) in cooled dichloromethane cooled was added EDC·HCl (300 mg) in dichloromethane. The mixture was stirred for 5 minutes before adding DMAP and a solution of pure capsaicin (200 mg) in dichloromethane. The reaction mixture was stirred at room temperature under a nitrogen atmosphere. Then the solvents are evaporated under reduced pressure and the crude was taken up in a mixture of EtOAc/Et2O and water. The organic layer was successively washed with 0.5 M NaOH, brine, 0.5 M HCl & brine, dried, filtered through a silica gel pad and evaporated under reduced pressure to afford the Boc-protected derivative. The oil was taken up in dichloromethane and EtOAc, the solution cooled before adding 3M HCl in CPME under nitrogen. The reaction mixture was stirred at room temperature for 5 hours, still under an inert atmosphere. The precipitate that formed was filtered, washed with dichloromethane and Et₂O, then triturated with Et₂O, acetone and Et₂O again to give capsaicin—beta-alanine.

Next, A slurry of Hyaluronic Acid (HA) molecular weight approx. 0.75 MDa; 125 mg) in a mixture of water and dioxane was stirred at room temperature until solubilised. A solution of N-hydroxysuccinimide (50 mg in water, then a solution of EDC·HCl 50 mg) in water (and finally a solution of the capsaicin—beta-alanine salt (50 mg) in water/dioxane were successfully added in that order with 5 minutes stirring between each addition. The reaction mixture was stirred at room temperature for 15 hours before the addition of NaHCO₃ as a solution in water. The reaction mixture was stirred again at room temperature for an additional 6 hours before the successive addition of AcOH in water then NaCl. The reaction mixture was stirred at room temperature and precipitated by adding ethanol. The mixture was stirred at room temperature for 30 minutes and filtered. The precipitate was washed with ethanol, twice with 85% ethanol in water, twice with ethanol and several times with Et₂O to give the HA-capsaicin composition. The HA-capsaicin composition solution is then passed through a 0.22 μm filter to sterilise, then the product is lyophilised and then refrigerated at 4-8° C.

Characterisation: The structure of the product, degree of conjugation and level of impurities are profiled using ¹H-NMR, mass spectrometry, with HPLC used to characterise sizing. These results are confirmed using other modalities such as FTIR spectroscopy, and DLS.

### 0.6 Synthesis of Capsaicin-Hyaluronic Acid Compositions Using a Beta-Alanine Linker

Hyaluronic Acid (HA) molecular weight approx. 0.75 MDa) was purchased from Contripro (Czech Republic), capsaicin was purchased from Merck (Ireland). All other regents were purchased from Merck (Ireland) unless noted. A 1% w/w solution of HA (1000 mg) is prepared in MES (2-(N-morpholino)ethanesulfonic acid) buffer. The HA is then activated using N-(3-dimethylaminopropyl)-N′-ethylcarbodiimide hydrochloride (EDC), at a molar ratio of 1:1.5 (HA:EDC). The reaction is carried out at pH 4.5-5.5 and room temperature for 30 minutes with gentle stirring. Capsaicin (1000 mg) is then dissolved in a small amount of DMSO (anhydrous dimethyl sulfoxide).

A bifunctional linker, beta-alanine, is dissolved in anhydrous DMSO and activated with EDC at a molar ratio of 1:2 (linker:EDC). The reaction is carried out at room temperature for 30 minutes with gentle stirring. The activated HA solution is mixed with the activated linker solution in a 1:2 molar ratio, and NHS is added at a molar ratio of 1:1 to improve the efficiency of the reaction. The reaction mixture is incubated at room temperature for 2-4 hours with gentle stirring. This results in an amide bond being formed between the carboxylic acid group of HA and the amine group of beta-alanine. Next the capsaicin solution is added to the mixture (1:5 mass ratio). The reaction mixture is allowed incubate at room temperature for 6-8 hours with gentle stirring. This resulted in an ester bond being formed between the carboxylic acid group of beta-alanine and the phenol group of capsaicin.

The resultant solution is purified using dialysis using DMSO and then deionised water (3 days each; dialysis membrane MWCO: 3000 Da). The HA-capsaicin composition solution is then passed through a 0.22 μm filter to sterilise, then the product is lyophilised and then refrigerated at 4-8° C.

Characterisation: The structure of the product, degree of conjugation and level of impurities are profiled using ¹H-NMR, mass spectrometry, with HPLC used to characterise sizing. These results are confirmed using other modalities such as FTIR spectroscopy, and DLS.

### 0.7 Synthesis of Capsaicin-Hyaluronic Acid Compositions Using a Beta-Alanine Linker

Initially capsaicin—beta-alanine compositions are synthesised as follow:

To a solution of N-(tert-Butoxycarbonyl)-β-alanine (200 mg) in cooled dichloromethane cooled was added EDC·HCl in dichloromethane. The mixture was stirred for 5 minutes before adding DMAP and a solution of pure capsaicin (200 mg) in dichloromethane. The reaction mixture was stirred at room temperature under a nitrogen atmosphere. Then the solvents are evaporated under reduced pressure and the crude was taken up in a mixture of EtOAc/Et2O and water. The organic layer was successively washed with 0.5 M NaOH, brine, 0.5 M HCl & brine, dried, filtered through a silica gel pad and evaporated under reduced pressure to afford the Boc-protected derivative. The oil was taken up in dichloromethane and EtOAc, the solution cooled before adding 3M HCl in CPME under nitrogen. The reaction mixture was stirred at room temperature for 5 hours, still under an inert atmosphere. The precipitate that formed was filtered, washed with dichloromethane and Et₂O, then triturated with Et₂O, acetone and Et₂O again to give capsaicin—beta-alanine.

Next, A slurry of Hyaluronic Acid (HA) molecular weight approx. 0.75 MDa; 250 mg) was dissolved in water and was stirred at room temperature until full solubilisation, before the addition of dioxane. The pH was adjusted with NaOH 1M, before the addition of the capsaicin—beta-alanine composition (100 mg) in a 1:1 H₂O/Dioxane mixture. the pH was adjusted with NaOH 1M before adding DMTMM (100 mg) in a 2:1 H₂O/Dioxane mixture with two drops of NaOH. The solution was stirred at room temperature, resulting in a homogenous and clear mixture. A second aliquot of the capsaicin—beta-alanine composition (100 mg) in H2O/Dioxane and DMTMM was added, and the pH was adjusted to 6-7 adding NaOH 1M. The reaction was stirred for 23 hours, resulting in a cloudy solution.

NaCl in H₂O was added, resulting in the formation of a gel-like solution. The solution was stirred at room temperature for 15 minutes, before the addition of EtOH. The supernatant was removed, and the precipitate was washed with EtOH, EtOH 85% in water, Acetone and Et₂O to afford the product. The HA-capsaicin composition solution is then passed through a 0.22 μm filter to sterilise, then the product is lyophilised and then refrigerated at 4-8° C.

Characterisation: The structure of the product, degree of conjugation and level of impurities are profiled using ¹H-NMR, mass spectrometry, with HPLC used to characterise sizing. These results are confirmed using other modalities such as FTIR spectroscopy, and DLS.

### 0.8. Synthesis of Resiniferatoxin-Hyaluronic Acid Compositions Using a Beta-Alanine Linker

A similar approach to that used in the example 0.5-0.7 above is used to conjugate resiniferatoxin (RTX) with HA using a beta-alanine linker. 1000 mg of HA was conjugated to 30 mg of RTX using the process described above.

### 0.9 Synthesis of Capsaicin/Resiniferatoxin-Hyaluronic Acid Compositions Using Other Linkers

A similar approach to that used in synthesis examples 0.5-0.7 above is used to conjugate capsaicin or resiniferatoxin (RTX) with HA using linkers which varied in length. These linkers included glycine, 5-aminovaleric acid (5 bonds long); (8-amino-3,6-dioxaoctanoic acid) (9 bonds long); (12-amino-4,7,10-trioxadodecanoic acid) (13 bonds long).

### 0.10 Synthesis of Cross-Linked Capsaicin-Hyaluronic Acid Compositions.

The compositions produced in examples 0.1-0.9 are further cross-linked using BDDE (1,4-butanediol diglycidyl ether) as follows.

A 2% (w/v) solution of a given composition is made in phosphate buffered saline (PBS; PBS: NaCl, 9 mg·mL⁻¹; KH₂PO4, 0.03 mg·mL⁻¹; Na₂HPO₄·2H₂O, 0.14 mg·mL⁻¹; pH 7.0). The solution is stirred until dissolution was complete, with NaOH added dropwise to adjust the pH to 8.0. Next BDDE is added at a concentration of 0.5% v/v and stirred for 2-4 hours at room temperature. After the crosslinking reaction is complete, the solution is dialysed against deionised water (3 days each; dialysis membrane MWCO: 3000 Da) in order to remove the residual BDDE and other impurities. The HA-capsaicin composition solution is then passed through a 0.22 μm filter to sterilise, then the product is lyophilised and then refrigerated at 4-8° C.

Characterisation: The structure of the product, degree of conjugation and level of impurities are profiled using ¹H-NMR, with HPLC used to characterise sizing. The level of BDDE in the product is to be assessed using LC-MS. These results are to be confirmed using other modalities such as FTIR spectroscopy, and DLS.

The cross-linking protocol is repeated using BDDE at concentrations of 0.1%, 0.2%, 1%, 2%, 5%, and 10% v/v—in order to adjust the level of crosslinking.

### Physicochemical Characterisation of the Polymer—Ion Channel Modulator Composition.

This details some of the characterisation tests for an exemplary final product—composition A, which is hyaluronic acid (750 kDa molecular weight) with capsaicin conjugated at 20% w/w using esterification between the —COOH groups of HA and the phenol group of capsaicin (cf. example B0.7 above). Similar characterisation is done for the other examples listed above.

### Particle Size Analysis:

The HA-capsaicin composition may form particles (colloidal particles or micelles) when in liquid media. An aqueous solution of HA-capsaicin at 1 mg/ml in deionised water is prepared with the haemodynamic diameter of the particles measured using dynamic light scattering with a He—Ne laser beam at a wavelength of 633.8 nm (Malvern Zetasizer Nano ZS90), with three replicates with a He—Ne laser beam at a wavelength of 633.8 nm. The results show the particles to follow a size distribution ranging from 10¹-10⁵ nm.

### Zeta-Potential Analysis:

The zeta-potential of the HA-capsaicin composition at pH 7.4, 25° C. is carried out using a Malvern Zetasizer Nano ZS90, and compared to HA alone, with three replicates.

The mean zeta potential of HA (FIG. 1A) was recorded at −23.2 mV, and the mean zeta potential of composition A (FIG. 1B) was recorded at −30.4 mV. These results prove that composition A is highly anionic and when compared with Hyaluronic acid, the composition has a higher negative value. A zeta potential of range+/−60 mV indicates stability in the case of colloid systems. In the case of composition A, while conjugation with capsaicin may theoretically be expected to reduce the negative charge the opposite effect was surprisingly seen to occur. This increased negative value compared to HA indicates compaction of the molecule with unexpectedly increased charge density (Note: Composition A detailed in Table 2a.1).

### Fourier Transform Infrared Spectroscopy (FTIR)

FTIR spectra of HA, capsaicin, and the HA-compositions in powder form is performed using a SHIMADZU-FTIR-8300 machine over a range from 4000-400 cm-1. All spectra are recorded at a 20-scan data accumulation at a resolution of 4 cm⁻¹. This modality allowed analysis of the functional groups of the tested materials—the spectra of the HA-capsaicin composition will show the presence of peaks characteristic of both HA and capsaicin, confirming synthesis. For example, a peak at about 3300 cm⁻¹ is characteristic of a phenol —OH and would not be present in unconjugated HA.

### ¹H Nuclear Magnetic Resonance (NMR)

NMR spectroscopy was carried out using an Agilent DD2 NMR 600 MHz. This modality is used to confirm synthesis—for example aromatic protons of capsaicin are expected, and seen, at approximately 6.6-7.1 ppm. NMR spectroscopy is further used quantitatively to confirm the desired 20% conjugation is achieved (based on integration of the peaks). Finally, NMR is also used to characterise the purity of the compositions—peaks correlating with reagents used in synthesis are expected to be seen to be reduced following purification and preliminary tests using NMR inform the purification protocol needed.

As shown in FIG. 2, The peaks associated with HA are recorded between 3.3 ppm and 4.6 ppm and appear as broad peaks. Peaks associated with the aromatic protons of capsaicin are still distinctive and recorded between 6.6 ppm and 6.9 ppm. The spectrum was acquired in D₂O+0.1M NaOH with addition of NaOH increasing proton mobility giving the best spectrum for HA.

¹H-NMR spectroscopy was used to assess the percentage of conjugation of beta-alanine comparing the integrations of the carboxylic protons (CH₂COO) of HA and the methylene protons of capsaicin.

### Polymer Stability/Degradation

Composition A samples degraded using Streptomyces derived hyaluronidase in an accelerated enzymatic study. The commercial HA product Durolane® (commerical HA) used as a control. The measurement of the released N-acetyl glucosamine (NAG) from composition A is performed according to the Reissig et al. method (Reissig J L, Strominger J L, Leloir L F. A modified colorimetric method for the estimation of N-acetylamino sugars. J Biol Chem 1955; 217:959-96).

Briefly, a diluted 1:10 Ehrlich's reagent (Sigma Aldrich) in acetic acid added to the tubes. The samples are vortexed and incubated for 20 min at 37° C., to develop a violet colour, proportional to the released NAG content in each sample. The tubes are centrifuged at 1000 g for 15 min to remove the gel fragments and the turbidity in the reaction. Then, the samples are read with a microplate reader at 585 nm wavelength against the blank prepared with the only phosphate buffer and the Ehrlich's reagent. The experiment is run for 96 hours.

The degradation profile of composition A is expected to compare favourably to the commercial product, with the latter indicated for use every 6 months.

Release Kinetics of Ion Channel Modulator from Polymer

The concentration of free capsaicin could be measured using a suitable ELISA kit. Composition A is solubilised in phosphate buffered saline at pH 7.4, 37° C., with agitation to model in-vivo conditions, at a 1 mg/ml concentration. Samples are taken periodically out to 28 days with the samples tested using ELISA to measure the capsaicin concentrations and capture the release kinetics. Capsaicin release is gradual and sustained over the time course of the experiment as the concentration in the samples built up gradually over time. (Note: Composition A detailed in Table 2a.1).

### Administration Test (In-Vitro and In-Vivo)

The present invention provides a solution, where covalent binding and optionally additionally mixing linking of ion channel modulators to the polymer provides a long-lasting effect when used for the modulation of nerve cells to provide for example pain relief. Moreover, the present invention provides a solution to reduce toxicity and safety signals related to the ion channel modulator.

In the case of pain relief and in the case of TRPV1 agonists, the present invention advantageously modulates ion flow into nerve cells and reduces the ability of cells to generate action potentials while minimising the initial excitation phase characteristic of these modulators. This reduction in action potential while minimising initial excitation translates into pain relief with a reduction in the initial acute period of sensitisation (i.e., initial period of acute pain) when applied to pain sensing neurons. In addition, the conjugation results in a depot of ion channel modulator which would be released over time in-vivo resulting in a profile within the therapeutic range and with an extended duration of action.

### Our Research Indicates:

1. Benefit of a sustained release approach in improving efficacy, safety, and duration of effect;
2. Reduction in initial excitation by using a lower dose;
3. Extended pain relief using a sustained dose.

### 2a: In-Vitro Assessment—Efficacy in Single Cell Patch Clamp

### Materials and Methods

All chemical, cell culture media and reagents are purchased from Sigma-Aldrich (Ireland), Fisher Scientific, Lonza, Abcam, and Alomone labs unless otherwise stated.

### Cell Culture

HEK293-TRPV1 cells purchased from Amsbio. Cells are cultured in DMEM medium supplemented with 10% foetal bovine serum (FBS), 2 mM glutamine, 1% Pen/strep and 1% non-essential amino acids. Cells are fed every two days and cultured at a density of 0.5 million/ml in a petri dish one day before the experiment.

### Single Cell Patch Clamp

Efficacy of exemplary compositions (Table 2a.1) is tested using an automated patch-clamp system to measure and screen the effect of control materials and test compositions on the modulation of current in HEK293-TRPV1 cells. TRPV-1 activation elicits an initial increase in current inflow into cells, followed by a desensitisation and reduction in current. The former is associated with an acute increase in pain in-vivo, while the reduction in current is translates into a reduction in pain in-vivo. Single-cell patch-clamp recording on Patchliner will be performed using an extracellular solution containing 140 mM NaCl, 4 mM KCL, 2 mM CaCl₂, 1 mM D-glucose monohydrate, 10 mM Hepes at pH 7.4 and osmolarity of 298 mOsm, and an intracellular solution containing 10 mM EGTA, 10 mM Hepes, 10 mM KCL, 10 mM NaCl, 110 mM KF at pH of 7.2 and osmolarity of over 280 mOsm. The Patchliner system is a fully automated, robust whole-cell patch clamp device, ideal for ion channel biophysics and mechanism of action and more sophisticated assays, including heat activation of TRPV1 channels.

The recording are made using an EPC10 patch clamp amplifier from HEKA. Whole-cell-induced currents are recorded in the voltage clamp mode in a holding potential of −60 mV at the baseline and in the presence of each test composition. The compositions induce an inward current of magnitude related to the concentration. The current amplitudes will be plotted against concentration. Using this approach, the half-maximal effective concentration (EC₅₀) of the compositions will be calculated, thus generating a drug-response curve. Data analysis will be performed using Patchmaster and IgorPro.

TABLE 2a.1
Compositions tested in the in-vitro patch clamp study.

| Composition code | Composition | Concentration Range | |
|---|---|---|---|
| A | HA (MW: 500-1000 kDa) | 0.001-3.0 | μg/ml |
| | conjugated to Capsaicin | | |
| | (CAP) at 15-25% w/w. | | |
| C | HA (MW: 500-1000 kDa) | 0.0001-3.0 | μg/ml |
| | conjugated to | | |
| | Resiniferatoxin (RTX) at | | |
| | 1-5% w/w | | |

### Results

Different combinations of compositions (A or C) are predicted to have two significant modes of action: 1) The current amplitude of activated HEK-TRPV1 cells will reduce with the application of the composition compared to free ion-channel modulator, and critically below the value expected if the ion channel modulator was not conjugated. 2) The cumulative release of ion channel modulator from HA conjugate will cause continuous activation TRPV1, leading to eventual reversible neuronal desensitisation. This prolonged release of ion channel modulator from the HA polymer will give prolonged neuronal desensitisation, translating into a sustained reduction in pain in-vivo.

### Conclusion

The TRPV1 ion channel in sensory neurons is a non-selective cation channel. When activated by agonists such as capsaicin and RTX, sodium and calcium ions flow through the channel, depolarising the nociceptive neurons, leading to action potential firing and inducing pain, followed by reversible desensitisation of nociceptive neurons. Slow and sustained release of capsaicin and RTX from the HA composition facilitates a level of TRPV1 activation below the level seen with free agonist (thus avoiding the acute pain sensation), with the cumulative effect inducing TRPV1 desensitisation and pain relief.

2b. In-Vitro Assessment—Release Kinetics of Ion Channel Modulator from Polymer

### Materials and Methods

Cells (HEK-293-TRPV1) are cultured and used on single cell patch clamp as described in Example 2a. Exemplary compositions A and B (Table 2b.1) are solubilised in phosphate buffered saline at pH 7.4 at a concentration of 1 mg/ml and stored at 4° C. (to assess stability), and at 37° C. with agitation (to model in-vivo conditions). Samples are taken periodically out to 16 days with the samples tested using patch clamp and free capsaicin assayed using a standard curve. Hence capsaicin release over the time course of the experiment is quantified.

TABLE 2b.1
Compositions tested in the in-vitro patch clamp study

| Composition | |
|---|---|
| code | Composition |
| A | HA |
| | (MW: 500-1000 kDa) conjugated to Capsaicin |
| | (CAP) at 15-25% w/w. |
| B | HA |
| | (MW: 500-1000 kDa) conjugated to Capsaicin |
| | (CAP) at 1-5% w/w. |

### Results

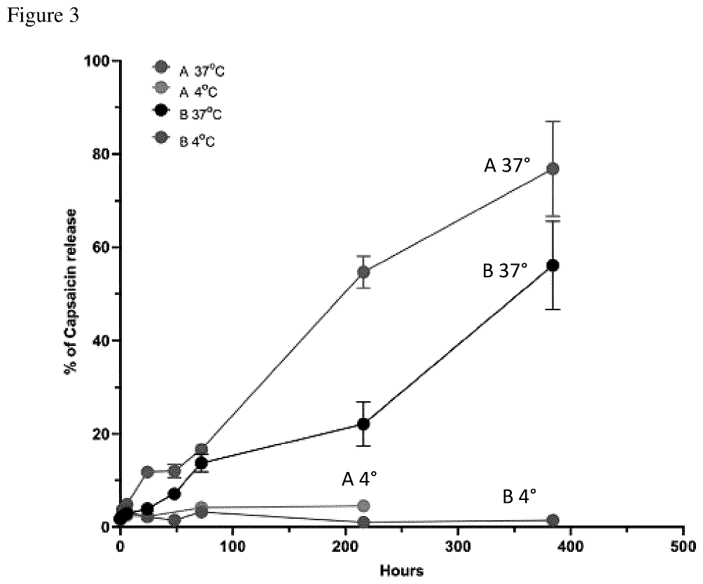

The results are shown in FIG. 3. The results show the compositions to have good stability at 4° C. with no significant capsaicin release over a >7-day time-course. Both Compositions A and B show release of capsaicin over 12.5% of the conjugated load by day 3 at 37° C. with agitation, and over 50% by day 16. Composition A shows faster release kinetics compared to Composition B.

### Conclusions

The exemplary Compositions A and B tested show good stability at 4° C., and reliable capsaicin release over the 16-day time course under modelled in-vivo conditions of 37° C. with agitation. Neither composition shows a burst release. The profile of kinetic release in-vitro correlates with the expected behaviour and benefits in-vivo of a minimisation of initial excitation with an effective initial lower dose compared to a bolus, and with pain relief provided by the depot of ion-channel modulator released over time within the therapeutic range and with an extended duration of action.

### 2c: In-Vitro Assessment—Efficacy on Chondrocyte Biomarkers

### Materials and Methods

All chemical, cell culture media and reagents are purchased from Sigma-Aldrich (Ireland), Fisher Scientific, Lonza, Abcam, and Alomone labs unless otherwise stated.

### Cell Culture

Normal and human osteoarthritis (OA) chondrocytes are purchased from Sigma-Aldrich. The cells are cultured in a chondrocyte basal growth medium. Cells are fed every two days and seeded at a density of 10⁵ cells in a 24-well plate one day before the experiment.

### Quantitative Polymerase Chain Reaction and Immunocytochemistry

The pattern of pro and anti-inflammatory proteins changes in OA. These changes are both a result of the disease and contribute to the pathology. Proinflammatory proteins include RAGE and IL6, while anti-inflammatory include IL-4 and Collagen II. The patterns of expression of these biomarkers can be measured using quantitative polymerase chain reaction (qPCR) and immunocytochemistry.

Following the treatment of the OA chondrocytes with different compositions, the expression of markers are examined at 24, 48, 72 hours at both gene and protein levels. To measure the expression of the markers at the gene level, RNA will be extracted using RNeasy Mini Kit (Qiagen). RNA (1 μg) will be used to generate complementary DNA using SuperScript III (Invitrogen).

Reverse transcript (RT)-PCR is performed using the Fast Start Universal SYBR Green Master (Roche) following the manufacturer's recommendations in a StepOne™ Real-Time PCR System (Thermofisher). Melting curve analysis is employed to confirm PCR specificity. Each reaction is run three times. Relative expression of the genes is quantified using the ΔCt method.

The expression of a number of the markers is assessed at the protein level using immunocytochemistry. Following the application (24, 48, 72 hours) of the test compositions (Table 2a.1, Table 2b.1), OA chondrocytes will be fixed using 4% paraformaldehyde for 10-20 minutes at room temperature. After blocking the cells with 0.1% bovine serum albumin (Sigma) in PBS for 30 minutes at room temperature, cells will be incubated with the appropriate primary antibody overnight at 4° C. Secondary antibodies will be applied for 1 hour at room temperature, and cell nuclei will be labelled with DAPI (Invitrogen). The expression of the markers will be quantified using an operetta fluorescent microscope.

### Conclusion

The changes in pattern of expression of representative pro and anti-inflammatory biomarkers in the presence or absence of therapeutic will indicate of the therapeutic has a beneficial secondary mode of action. HA is known to have anti-inflammatory effects through interaction with chondrocytes, and these cells re also known to have ion channels.

### 2d: In-Vitro Assessment—Safety

### Materials and Methods

All chemical, cell culture media and reagents are purchased from Sigma-Aldrich (Ireland), Fisher Scientific, Lonza, Abcam, and allomone labs unless otherwise stated.

### Cell Culture

For examining the safety of the compositions in neurons, ND7/23 cell line is a DRG-like cell line which will be transfected with TRPV1. The TRPV1 plasmid will be purchased from Origene. Cells will be cultured in Low glucose medium supplemented with 10% FBS and 1% Pen/strep. Cells will be fed every two days and seeded at a density of 10⁴ cells in a 96-well plate one day before the experiment.

Normal and human osteoarthritis (OA) chondrocytes are purchased from Sigma-Aldrich. The cells will be cultured in a chondrocyte basal growth medium. Cells will be fed every two days and seeded at a density of 10⁴ cells in a 96-well plate one day before the experiment.

### Cell Viability/Safety Assessment

Metabolic activity is assessed using the Alamar blue assay (Thermo Fisher Scientific, UK) after 24 and 48 hours in culture, according to the manufacturer's protocol. Alamar blue is a cell proliferation assay that provides a rapid, sensitive, and economical way to quantitatively measure cell proliferation and cytotoxicity in various human and animal cell lines. Resazurin is an oxidation-reduction (REDOX) indicator that undergoes a colourimetric change in response to cellular metabolic reduction. The reduced form, resorufin, is pink and highly fluorescent, and the fluorescence intensity is proportional to the number of living cells respiring. Alamar blue is a direct indicator of cell health; it detects oxidation levels during respiration, quantitatively measuring cell viability and cytotoxicity. The safety of all compositions is assessed with Alamar blue assay in ND7/23-transfected TRPV1, normal and OA chondrocytes in the concentration range shown in Table 2c.1.

TABLE 2c.1
Compositions tested in the in-vitro safety

| Composi- | | | |
|---|---|---|---|
| tion | | Concentration | |
| code | Composition | Range | |
| A | HA (MW: 500-1000 kDa) conjugated to | 0.001-200 | μg/ml |
| | Capsaicin (CAP) at 15-25% w/w. | | |
| C | HA (MW: 500-1000 kDa) conjugated to | 0.0001-200 | μg/ml |
| | Resiniferatoxin (RTX) at 1-5% w/w | | |

### Results

A dose-response curve of toxicity across the concentration range is generated for the compositions cytotoxicity in both neurons and chondrocytes. The results show the conjugated forms to be less toxic at a given concentration compared to free ion channel modulator. In addition, the therapeutic window (based on patch clamp efficacy) is wide.

### 3a: In-Vivo Assessment—Näive Animals

### Objective:

To investigate the safety of exemplary composition A (see example B0.7 above) at different doses in naïve animals. Composition A has a TRPV1 agonist (capsaicin) as the ion channel modulator—the present invention will minimise the initial excitation phase characteristic of these modulators. This experiment will hence demonstrate the superiority of the conjugation chemistry in lowering acute sensitisation. This initial excitation phase is most acute in the first hours after intra-articular administration of free TRPV1 agonist in rats.

### Materials and Methods

Details on the compositions, chemicals, induction agents, and treatments to be used in this study are summarised in Tables 3a.1.

TABLE 3a.1
Treatments to be used in the in-vivo study.

| | Ion | | | |
|---|---|---|---|---|
| Treatment | Channel | | | |
| Name | Modulator | Solvent | Concentration | Notes |
| Vehicle | | PBS | N/A | |
| CAP | Capsaicin | PBS | 10 μg/50 μL | |
| HA | | PBS | 1 μg/50 μL | HA component of |
| | | | | Composition A |
| CAP + HA | Capsaicin | PBS | 1.25 μg/50 μL | Unconjugated components of |
| | | | | Composition A |
| Composition | Capsaicin | PBS | 1.25 μg/50 μL | Tested at three strengths with |
| A | | | 6.25 μg/50 μL | loading of 10 μg, 50 μg, |
| | | | 12.5 μg/50 μL | 100 μg of capsaicin. |

### Procedure

Naïve rats (Sprague Dawley, female, 8 weeks old) under brief anaesthesia (isoflurane) were administered into the knee through intra-articular injection using a 0.5 ml insulin syringe (30G, 8 mm) needle at a volume of 50 μL.

### Pain-Related Behaviour Tests

Pain related behaviours were assessed using:
Von Frey (vF)—secondary hypersensitivity on the ipsilateral plantar surface measured using an electronic von Frey anaesthesiometer (eVF). Pressure is applied on the plantar surface until a response (withdrawal, flinching, licking) observed.
EthoVision scored observation in the first 15 minutes after treatment—pain and nocifensive behaviours including immobility measured in terms of time for each treatment.

These behaviours are measured at baseline, and in the immediate hours following treatment.

### Results:

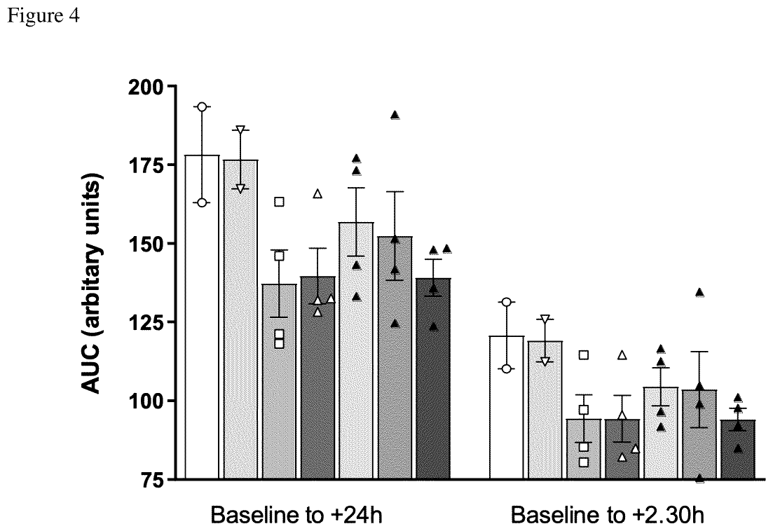

Following administration of the test materials von Frey testing was performed measuring paw withdrawal threshold (PWT) on the ipsilateral paw. The results (FIG. 4) show exemplary composition A has a higher PWT compared to both capsaicin and the mixture group demonstrating the benefit of conjugation of capsaicin in allowing loading of up to ×10 the levels of capsaicin without the same level of acute sensitisation (i.e. pain).

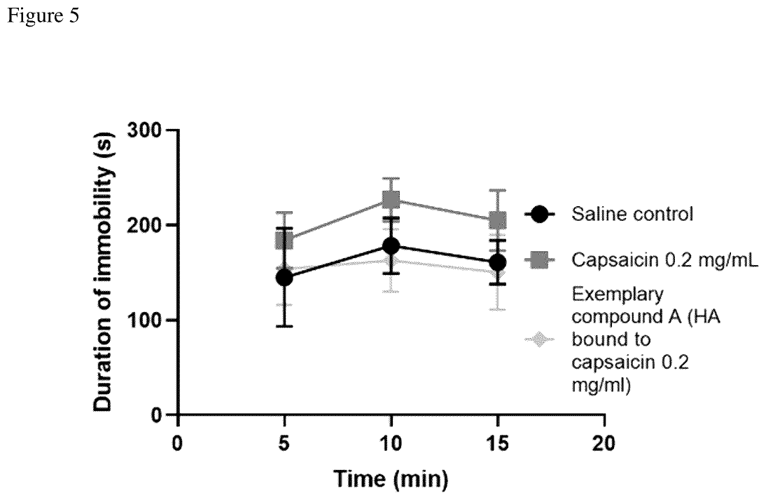

In addition, immobility as a nocifensive behaviour was measured for the first 15 minutes following treatment. The results (FIG. 5) show exemplary composition A leads to a duration of immobility (as a measure of pain) equivalent to that of control, and less than that of free capsaicin. This result demonstrates the benefit of conjugation of capsaicin in reducing the initial acute period of sensitisation.

### 3b: In-Vivo Assessment—MIA Model of Osteoarthritis

### Objective:

To investigate the efficacy and safety of different compositions and at different doses in the rat monosodium iodoacetate model of knee osteoarthritis (OA). The MIA model is a standard preclinical model for mimicking joint disruption in OA and assessing subsequent OA-related chronic pain behaviours.

### Materials and Methods

Details on the compositions, chemicals, induction agents, and treatments to be used in this study are summarised in Tables 3b.1, 3b.2, 3b.3, and 3b.4 respectively.

TABLE 3b.1
Compositions to be tested in the in vivo study

| Composition | |
|---|---|
| code | Composition |
| A | HA (MW: 500-1000 kDa) conjugated to Capsaicin |
| | (CAP) at 15-25% w/w. |
| C | HA (MW: 500-1000 kDa) conjugated to Resiniferatoxin |
| | (RTX) at 1-5% w/w |

TABLE 3b.2
Chemicals to be used in the in-vivo study

| Chemical | Abbreviated | | | Batch |
|---|---|---|---|---|
| Name | Name | Constituents | Company | Number |
| Phosphate | PBS | Sodium chloride 9 mg; | Sigma | N/A |
| Buffered | | Potassium Dihydrogen | Aldrich | |
| Saline | | Phosphate 0.03 mg; | (Merck) | |
| | | Disodium Hydrogen | unless | |
| | | Phosphate Dihydrate | stated | |
| | | (Merck) 0.14 mg; Water | | |
| | | for Injection q.s. 1 ml | | |
| Sodium | MIA | Sodium iodoacetate ≥ | Sigma | SLBZ7569 |
| mono- | | 98% | Aldrich | |
| iodoacetate | | | (Merck) | |

TABLE 3b.3
Induction agents to be used in the in-vivo study.

| Induction Agent | | | |
|---|---|---|---|
| Name | Solute | Solvent | Concentration |
| Vehicle | N/A | PBS | N/A |
| MIA | MIA | PBS | 40 mg/ml (2 mg/50 μL) |

TABLE 3b.4
Treatments to be used in the in-vivo study.

| Treatment | Ion Channel | | | | |
|---|---|---|---|---|---|
| Name | Modulator | Solvent | Concentration | | Notes |
| Vehicle | | PBS/DMSO | N/A | | |
| CAP | Capsaicin | PBS/DMSO | 0.2 | mg/ml | Dose administered = |
| | | (1:1) | | | 10 μg/50 μL |
| RTX | Resiniferatoxin | PBS/DMSO | 0.03 | mg/ml | Dose administered = |
| | | (1:1) | | | 1.5 μg/50 μL |
| A | CAP | PBS | 5 and 10 | mg/ml | HA Conjugated |
| | | | | | to CAP |
| | | | | | Two different |
| | | | | | concentrations |
| | | | | | used - compare |
| | | | | | dose effects |
| C | RTX | PBS | 5 and 10 | mg/ml | HA Conjugated |
| | | | | | to RTX |
| | | | | | Two different |
| | | | | | concentrations |
| | | | | | used - compare |
| | | | | | dose effects |

### Animals

The study will be conducted using adult, male Sprague-Dawley rats (8-10 weeks old, 200-250 g, Charles River UK). The animals will be maintained under standard laboratory conditions (temperature 22±2° C., relative humidity 45-65%, 12:12 h light/dark cycle with lights on from 08:00-20:00 h) and housed in groups of 2-3 rats per cage in standard plastic bottom cages containing 3Rs basic bedding (3Rs Laboratory, UK), nesting material (Sizzle-Pet, LBS Biotechnology, UK), and a plastic tube. Food and water will be available ad libitum throughout the study. The rats will be acclimatised for a minimum of 7 days after arrival into the unit. No prophylactic or therapeutic treatment will be administered during this period.

### Intra-Articular Injection

Under brief anaesthesia (2-3% isoflurane, 0.8 L/min O₂), agents to induce the model (Table 3.b3) or assigned treatments (Table 3b.4) will be administered into the knee through intra-articular injection using a 0.5 ml insulin syringe (30G, 8 mm) needle at a volume of 50 μL. The needle will be inserted perpendicularly through the infrapatellar ligament into the intra-articular space of the left tibiofemoral (knee) joint, with the contents of the syringe dispensed into the joint.

### Pain-Related Behavioural Tests

Pain-related behaviours will be assessed in the animals using the static weight bearing (SWB) and pinch tests.

### Weight Bearing Test to Assess Weight Distribution Asymmetry

The SWB test will measure spontaneous pain on joint loading. An Incapacitance Tester (Linton Instrumentation, UK) is used for the test. The device has an angled plexiglass chamber and two separate force plates at the base of the chamber. After calibrating the plates with a 50 g mass, the animal will be introduced into the chamber and allowed to rest with its forepaws on the plexiglass and the hind paws on the force plates. The force exerted (in grams) by each hind paw is measured over a period of 3 s. The distribution of weight borne on the hind limbs is reported as the percentage weight borne on the ipsilateral (injured/left) hind limb, calculated using the following equation:

$$Percentage weight bearing = \frac{Weight on ipsilateral hind limb}{Weight on contralateral + ipsilateral hind limbs} \times 100$$

Three successive trials will be performed for each rat and the mean percentage weight borne on the ipsilateral hind limb will be calculated. A decrease in the percentage weight borne in the ipsilateral hind limb indicates weight bearing deficit (or asymmetry) in the injured hind limb.

### Pinch Test to Assess Mechanical Hyperalgesia

The pinch test will measure mechanical hyperalgesia in the rat knee joint. A ‘rodent pincher’ algometer (Bioseb, France) is used for the test. A pair of calibrated forceps is used to apply pressure (mechanical stimulation) around the knee joint. The pressure applied is increased gradually at a constant rate until a pain-related response (brisk withdrawal or flinching of the knee, or vocalisation) is obtained. The maximum pressure (in grams) is recorded by a meter connected to the forceps and designated as the withdrawal threshold. The test is repeated 4 times for each rat to obtain mean withdrawal threshold. A decrease in the withdrawal threshold will indicate mechanical hyperalgesia in the injured knee joint.

### Study Design

There will be 8 experimental groups in this study with n=11 rats per group (Table 3b.5).

TABLE 3b.5
Experimental groups in the in vivo study.

| | | | Administration | Volume | |
|---|---|---|---|---|---|
| Group | Induction | Treatment | Route | (μL) | n |
| 1 | PBS (Sham) | PBS | i.a | 50 | 11 |
| 2 | MIA 2 mg | PBS | i.a | 50 | 11 |
| 3 | MIA 2 mg | CAP | i.a. | 50 | 11 |
| | | (0.2 mg/ml) | | | |
| 4 | MIA 2 mg | A | i.a. | 50 | 11 |
| | | (5 mg/ml) | | | |
| 5 | MIA 2 mg | A | i.a. | 50 | 11 |
| | | (10 mg/ml) | | | |
| 6 | MIA 2 mg | RTX | i.a. | 50 | 11 |
| | | (0.03 mg/ml) | | | |
| 7 | MIA 2 mg | C | i.a. | 50 | 11 |
| | | (5 mg/ml) | | | |
| 8 | MIA 2 mg | C | i.a. | 50 | 11 |
| | | (10 mg/ml) | | | |

Randomisation: After acclimatisation, animals will be tested for baseline behavioural responses on Days −3 and −1 (SWB and pinch tests) with respect to MIA injection. Two measurements will be performed for each behavioural test to obtain stable baseline responses, and averaged. Animals will then be randomly allocated to treatments groups resulting in 8 experimental groups. The experimenter will remain blind to group allocation.

Experimental protocol: The study will be conducted in 2 batches. Batch 1 will consist of 40 rats divided into 4 blocks (n=10 rats per block). In Batch 2, there will be 48 animals that will be divided into 5 blocks (n=10, 10, 8, 10, and 10 per block). Each block will have a random allocation of animals from the experimental groups. The time course of each animal will be the same, with blocks staggered by one day.

Following baseline pain testing, rats will receive an i.a. injection of MIA (40 mg/ml or 2 mg/50 μL) under brief anaesthesia to induce GA-like lesions in the left knee joint on Day 0. Sham animals will receive an i.a. injection of saline (0.9% NaCl) into the left knee under the same conditions. The animals will be allowed to recover in their home cages. After 14 days, another i.a. injection of the assigned treatment (Table 5) will be administered into the knee under brief anaesthesia. Rats will be assessed in the SWB and pinch tests (in that order) for pain-related behaviours in the knee joint on Days 7, 10, 14, 21, 28, 42, 56, 70, and 98. On Day 14, SWB and pinch tests will be conducted at +30, +60, +120, +240, and +360 min post-treatment injection to assess any treatment-related acute pain sensitivity in the knee. Animals will be euthanised on Day 99 by decapitation.

### Statistical Analysis

The behavioural results will be analysed using repeated measures ANOVA (within subjects factor: time, between subjects factor: treatment) followed by, where appropriate, post hoc Tukey's test for pairwise comparisons. P<0.05 will be set as threshold for statistical significance.

### Expected Outcomes:

### Static Weight Bearing Test

The results of the SWB test will be expressed in percentage weight borne on the ipsilateral (left) hind limb. MIA-injected rats will display reduced weight borne on the ipsilateral hind limb compared to sham counterparts, indicating MIA-induced weight distribution asymmetry.

Treatments will be administered via i.a. injection into the knee on Day 14. Compared to vehicle-treated counterparts, MIA rats treated with free CAP or RTX will show increased weight bearing asymmetry at acute time points following administration on Day 14. CAP or RTX will improve weight bearing asymmetry in MIA rats at early time points with effects lasting for not more than 2 weeks post-treatment injection. In comparison, weight bearing asymmetry at acute time points on Day 14 will not be different between the MIA rats treated with the composition (A or C) and vehicle. Moreover, both compositions A and C will significantly improve weight bearing deficit in the MIA rats giving a sustained effect that will persist at the later time points.

### Pinch Test

MIA injection will reduce the withdrawal threshold in the knee compared to sham counterparts, indicating mechanical hyperalgesia in the knee joint.

I.a. injection of CAP or RTX will increase this mechanical hypersensitivity in the MIA rats at acute time points on Day 14, compared to vehicle-treated counterparts. CAP or RTX will increase withdrawal threshold to mechanical stimulus in MIA rats at early time points, indicating reduction in mechanical hyperalgesia. This CAP- or RTX-induced effect on mechanical hyperalgesia is expected to last for 1-2 weeks. In comparison, mechanical hypersensitivity at acute time points on Day 14 will be similar between the MIA rats treated with the composition (A or C) and vehicle. Both compositions A and C will improve mechanical hyperalgesia in the knee of MIA rats, giving sustained effects that will persist at the later time points.

### Conclusions:

The results from this in vivo study will indicate that:
I.a. injection of the current porotypes (compositions A and C) does not induce acute pain sensitivity as demonstrated by no effect on weight bearing deficit (SWB test) and mechanical hyperalgesia (pinch test) in MIA rats compared to vehicle-treated counterparts on the day of treatment administration.
The current compositions significantly improve OA-associated chronic pain behaviour in the SWB and pinch tests that persists at the later time points, indicating sustained, long-lasting analgesic effects.

What is claimed is:

1. A composition for treatment of pain, wherein the composition comprises a carrier, and a polymer covalently linked to at least one class of ion channel modulators selected from the group consisting of modulators of transient receptor potential cation channel subfamily V member 1 (TRPV1) channels, voltage gated sodium (Nav) channels, voltage gated calcium (Cav) channels, and combinations thereof; and optionally additionally at least one class of ion channel modulators being mixed with the polymer.

2. The composition of claim 1, wherein the composition comprises one or more TRPV1 modulators selected from the group consisting of capsaicin (e.g. (Z)-capsaicin or (E)-capsaicin), capsaicinoids, capsinoids resiniferatoxin (RTX), olvanil, C18-Nacetylethanomimes, MSP-3 and vanilotoxin.

3. The composition of claim 1, wherein the composition comprises one or more Nav modulators selected from the group consisting of lidocaine, bupivacaine, ropivacaine, tetracaine, and tetrodotoxin.

4. The composition of claim 1, wherein the composition comprises one or more Cav modulators selected from ziconotide and gabapentinoids including gabapentin and pregabalin.

5. The composition of claim 4, wherein the polymer is a biocompatible polymer and is selected from the group consisting of poly-ethylene glycol (PEG), poly(lactic-co-glycolic acid) (PLGA), polylactic acid, polysaccharides, silica, hyaluronic acid (HA) or an ester or salt thereof, polycaprolactone (PCL), polyamides and poly(ester-amide)s, polyurethanes, polyanhydrides, polyvinyl alcohol, chitin, cellulose, alginic acid or alginate, poly(hydroxybutyrate) (PHB), starch and its modified polymers, dendrimers, collagen, gelatin, calcium hydroxyapatite, polyethylene glycol diacrylate, sodium hyaluronate, polyglycolide, poly-3-hydroxybutyrate, and chitosan; or combinations thereof.

6. The composition of claim 5, wherein the polymer is cross linked.

7. The composition of claim 5, wherein the polymer is HA or PEG, and molecular weight of the polymer is between 50 and 3000 kDa.

8. The composition of claim 1, wherein the polymer is covalently linked to the ion channel modulator directly or via a linker.

9. The composition of claim 8, wherein the linker is selected from the group consisting of beta-alanine, glycine, 5-aminovaleric acid; (8-amino-3,6-dioxaoctanoic acid); and (12-amino-4,7,10-trioxadodecanoic acid).

10. The composition of claim 1, wherein covalent link between the polymer and ion channel modulators is selected from the group consisting of ester bonds, amide bonds, carbamate bonds, carbonate bonds, and urethane bonds.

11. The composition of claim 1, wherein the polymer is covalently linked to the ion channel modulator in a in a mass:mass ratio in a range of 1:0.001 to 1:5 polymer to ion channel modulator.

12. The composition of claim 1, wherein the composition has sustained release of an ion channel modulator, and wherein release of ion channel modulator takes place in 0 to 12 months.

13. A composition for pain relief comprising:
the composition comprises a carrier;
a polymer selected from the group consisting of HA, PEG, and a combination thereof;
at least one TRPV1 modulator covalently linked to the polymer in a mass:mass ratio of 1:0.03,
and wherein the composition has a sustained release of the ion channel modulator.

14. The composition of claim 13, wherein the TRPV 1 modulator is capsaicin or resiniferatoxin.

15. The composition of claim 13, wherein the polymer is hyaluronic acid having molecular weight between 500 and 1000 kDa.

16. The composition of claim 13, wherein the composition is suitable for administration as an injection.

17. A method to release at least one ion channel modulator in a sustained and prolonged manner at a location of pain, said method comprising injecting a composition of claim 1 at the location of pain.

18. The method of claim 17, wherein the polymer is selected from the group consisting of HA, PEG, and a combination thereof;
and the ion channel modulator is TRPV1 modulator, preferably capsaicin or resineferitoxin or combination thereof covalently linked to the polymer in a mass:mass ratio of 1:0.03.

19. The method of claim 17, wherein an injection volume is 1-10 ml and the injection volume comprises from 1 μg to 150 mg of ion channel modulator per millilitre.

20. The method of claim 17, wherein the pain is osteoarthritis pain.
